(19) United States
(12) Patent Application Publication
(10) Pub. No.: US 2010/0266587 A1
(43) Pub. Date: Oct. 21, 2010

(54) Compositions and Methods to Treat Acute Myelogenous Leukemia

(75) Inventor: Karen McLachlan, SOLANA BEACH, CA (US)

Correspondence Address: LANDO & ANASTASI, LLP;B2047, ONE MAIN STREET, SUITE 1100, CAMBRIDGE, MA, 02142, US

(73) Assignee: BIOGEN IDEC MA INC., CAMBRIDGE, MA (US)

(21) Appl. No.: 12/761,853

(22) Filed: Apr. 16, 2010

Related U.S. Application Data
(60) Provisional application No. 61/170,551, filed on Apr. 17, 2009.

(51) Int. Cl. A61K 39/395; A61P 35/02

(52) U.S. Cl. 424/133.1; 424/144.1; 424/173.1

(57) ABSTRACT

Compositions and methods for treating or preventing a hematologic malignancy, such as AML, using an anti-alpha4 antibody are described.

### RELATED APPLICATIONS

This application claims the benefit of U.S. Provisional Application No. 61/170,551, filed Apr. 17, 2009, which is incorporated herein by reference in its entirety.

### BACKGROUND

Hematological malignancies are proliferative disorders that affect blood, bone marrow, and lymph nodes. They include leukemias, such as chronic lymphocytic leukemia (CLL) and acute myelogenous leukemia (AML), lymphomas and multiple myeloma.

### SUMMARY

The invention is based, in part, on the discovery that anti-alpha4 antibodies can block VLA-4 mediated adhesion of hematologic cell lines (including cell lines of acute myelogenous leukemia (AML)) to VCAM-1 and fibronectin, as well as to bone marrow stromal cells. While not wishing to be bound by theory, this activity can disrupt cell survival signaling pathways and increase sensitivity of cells to cytotoxic agents. Thus, methods of treating hematological malignancies, such as AML, or for decreasing resistance to cytotoxic agents using anti-alpha4 antagonists are provided.

In one aspect, a method of treating a patient having a hematological disorder, e.g., a leukemia, such as acute myelogenous leukemia (AML) is provided. The method includes administering to the patient a therapeutically effective amount of a composition containing an antagonist of an interaction between an integrin with an alpha4 subunit (e.g., VLA-4) and a ligand for this integrin (e.g., VCAM-1). This antagonist can be an alpha4 integrin binding agent or an alpha4 integrin ligand binding agent. Typical agents include anti-VLA-4 or anti-alpha4beta7 antibodies (e.g., human, chimeric, and humanized antibodies and fragments thereof); anti-VCAM-1 antibodies (e.g., human, chimeric, and humanized antibodies and fragments thereof); and small molecule inhibitors of interactions of alpha4 subunit containing integrins with their ligands.

In one embodiment, the antagonist is an anti-alpha4 integrin antibody or antigen binding fragment thereof, e.g., a VLA-4 binding antibody, or antigen binding fragment thereof. The composition can be a pharmaceutical composition containing at least the therapeutically effective amount of VLA-4 binding antibody, and a pharmaceutically acceptable carrier.

In another embodiment, the anti-alpha4 binding antibody or antigen binding fragment thereof is a VLA-4 binding antibody or fragment thereof. In another embodiment, the anti-alpha4 antibody, or antigen binding fragment thereof, e.g., the VLA-4 binding antibody, is a human antibody, a chimeric antibody, a humanized antibody or an antigen-binding Fab, Fab′, F(ab′)2 or F(v) fragment of a human, chimeric or humanized antibody, or a modified antibody with more than two antigen binding sites (e.g., a bispecific antibody). In another embodiment, the antibody or antigen-binding fragment thereof is a monoclonal or monospecific antibody, a single chain antibody (e.g., a nanobody, such as a camel or a shark antibody (an IgNAR)), or an antigen-binding fragment of any of these types of antibodies.

In one embodiment, the antagonist is a small molecule inhibitor, such as an inhibitor described in WO 06/131200 or US2007/0004775, both of which are incorporated herein by reference.

In another embodiment, the composition is administered at a dosage so as to provide from about 0.1 to about 20 mg/kg body weight of the antibody or antigen binding fragment thereof.

In another embodiment, the anti-alpha4 antibody or antigen-binding fragment thereof, binds the alpha chain of VLA-4, and in yet another embodiment, the antibody or antigen-binding fragment thereof is a B epitope specific VLA-4 binding antibody or antigen-binding fragment thereof. In another embodiment, the antibody or antibody fragment is natalizumab, or an antigen binding fragment of natalizumab.

In one embodiment, the method of treating the hematological malignancy (e.g., AML) includes administering a second therapeutic agent in addition to the anti-alpha4 antibody. The second therapeutic agent can be, for example, a chemotherapeutic agent, such as (but not limited to) cytarabine (Ara-C), daunorubicin, idarubicin, etoposide, gemtuzumab ozogamicin, arsenic trioxide, or all-trans retinoic acid. The method of treating the hematological malignancy can also include a second therapeutic regimen, e.g., radiotherapy, in addition to the administration of the alpha4 antagonist.

Some embodiments are suitable for delivery to a subject, such as a human, e.g., a human patient, by subcutaneous (SC) or intramuscular (IM) delivery. A composition containing an anti-alpha4 antibody can also be suitable for intravenous (IV) administration, such as, when diluted into an acceptable infusion matrix (such as normal saline). The anti-alpha4 antibody can be natalizumab, for example.

In one embodiment, the anti-alpha4 antibody is a humanized monoclonal antibody, such as natalizumab. In another embodiment, the anti-alpha4 antibody is a variant of natalizumab. For example, in some embodiments, the light chain variable region of the antibody has an amino acid sequence that differs by one or more amino acid residues, but not more than 2, 3, 4, 5, or 6 amino acid residues of the light chain variable region of natalizumab, and/or the heavy chain variable region has an amino acid sequence that differs by one or more amino acid residues, but not more than 2, 3, 4, 5, or 6 amino acid residues of the heavy chain variable region of natalizumab. In some embodiments, some or all differences are conservative changes. In some embodiments, the anti-alpha4 antibody has CDRs equivalent to the CDRs of natalizumab, or the antibody binds the same or an overlapping epitope as natalizumab.

In another embodiment, the anti-alpha4 antibody has one or both of a light chain variable region having the amino acid sequence of SEQ ID NO:7 in U.S. Pat. No. 5,840,299, which is incorporate by reference herein, and a heavy chain variable region having the amino acid sequence of SEQ ID NO:11 in U.S. Pat. No. 5,840,299. In other embodiments, the VLA-4 antibody is a variant of one of these antibodies. For example, in some embodiments, the light chain variable region has an amino acid sequence that differs by one or more amino acid residues, but not more than 2, 3, 4, 5, or 6 amino acid residues from the sequence in SEQ ID NO:7 in U.S. Pat. No. 5,840,299, and/or the heavy chain variable region has an amino acid sequence that differs by one or more amino acid residues, but not more than 2, 3, 4, 5, or 6 amino acid residues as defined by SEQ ID NO:11 in U.S. Pat. No. 5,840,299.

In yet another embodiment, the anti-alpha4 antibody has one or both of a light chain amino acid sequence of SEQ ID NO:1 in Table 1-1, and a heavy chain amino acid sequence of SEQ ID NO:2 in Table 1-2. In other embodiments, the VLA-4 antibody is a variant of one of these antibodies. For example, in some embodiments, the light chain of the antibody has an amino acid sequence that differs by one or more amino acid residues, but not more than 2, 3, 4, 5, or 6 amino acid residues from the sequence of SEQ ID NO:1, and/or the heavy chain of the antibody has an amino acid sequence that differs by one or more amino acid residues, but not more than 2, 3, 4, 5, or 6 amino acid residues from the sequence of SEQ ID NO:2.

A “difference” in amino acid sequence, as used in this context, means a difference in the identity of an amino acid (e.g., a substitution of a different amino acid for an amino acid in SEQ ID NO:7 or 11 referred to above) or a deletion or insertion. A difference can be, for example, in a framework region, a CDR, a hinge, or a constant region. A difference can be internal or at the end of a sequence of protein. In some embodiments, some or all differences are conservative changes as compared to the recited sequence.

In one embodiment, the method allows for a gradual increase in the antibody dosage provided (dosage as used here refers to the amount of antibody provided in one, or in each of a defined small number, e.g., 2, administrations). This allows ramp-up of dosage and can allow monitoring of the patient for tolerance, adverse reactions, and the like as the dosage is increased. For example, the method can begin by providing natalizumab to the patient at one or more initial or relatively low dosages followed by providing natalizumab to the patient at a final, higher dosage. Typical initial dosages can be, e.g., 80%, 70%, 50%, 30%, 20% or 10% or less of the final higher dosage. Typical final dosages will vary based on the frequency of administration once steady state administration has been achieved. For example, some embodiments include final dosages of between 50 mg and 1200 mg per 28 days IV administration. Some embodiments include final dosages of between 10 mg and 1000 mg (e.g., 50 mg, 100 mg, 150 mg, 200 mg, 250 mg, 300 mg, 350 mg, 400 mg, 450 mg, 500 mg, 600 mg, 700 mg, 800 mg, 900 mg) (these dosages can be typical of approximately monthly administration). Other embodiments include final dosages of between 25 mg and 250 mg (e.g., 50 mg, 75 mg, 100 mg, 150 mg, 200 mg) (these dosages are typical of administration every two weeks). Therapeutic dosing can be determined by receptor saturation.

In some embodiments, the patient will receive one or a plurality of administrations, at one or a plurality of initial dosages. For example, in one embodiment, the patient will receive increasing dosages over a number of administrations. In some embodiments, the patient will receive 2, 3, 4, 5, 6, 7, or 8 administrations at one or more initial dosages prior to reaching the final dosage. For example, the patient will receive one or more administrations at a first initial dosage, and one or more administrations at a second higher initial dosage. In some embodiments, the patient is assessed after one or more administrations for symptoms, including adverse symptoms. In some embodiments, the patient is administered an increased dosage of natalizumab only after determining that the patient does not have an unacceptable adverse reaction to the previous dosage.

In some embodiments, that patient will receive an initial higher dose and then subsequence lower doses, e.g., as symptoms improve.

In one embodiment, the patient is administered an initial dose of the alpha4 antagonist (e.g., an alpha4 binding antibody) at the same time as receiving an initial dose of a chemotherapeutic agent or radiotherapy treatment. In another embodiment, the patient is administered an initial dose of the alpha4 antagonist after having a relapse of a hematological malignancy.

In another aspect, the invention features a method, e.g., a method of instructing a patient in need of an alpha4 antagonist therapy, how to administer a composition described herein for the treatment of a hematological malignancy. The method includes (i) providing the patient with at least one unit dose of a formulation of an antagonist, e.g., an anti-alpha4 antibody; and (ii) instructing the patient to self-administer the at least one unit dose intravenously. Another method, e.g., a method of treatment, includes (i) providing the patient with at least two unit doses of a formulation of alpha4 antagoinst; and (ii) instructing the patient to self-administer the unit doses intravenously, e.g., one dose at a time.

In one embodiment, the patient has a hematological disorder, such as a leukemia, e.g., acute myelogenous leukemia (AML), acute lymphoblastic leukemia (ALL), chronic lymphocytic leukemia (CLL), chronic myelogenous leukemia (CML), or hairy cell leukemia (HCL). In another embodiment, the patient has a lymphoma, such as Hodgkin's disease or Non-Hodgkin's lymphoma (either T- or B-cell type). In another embodiment, the patient has myelodysplastic syndrome (MDS), and in another embodiment, the patient has a myeloproliferative disease, such as polycythemia vera (also called PV, PCV or polycythemia rubra vera (PRV)), essential thrombocytosis (ET), or myelofibrosis. In yet another embodiment, the patient has amyloid due to light-chain disease, Waldenstroms macroglobulinemia, monoclonal gammopathy of unknown significance (MGUS), or plasma cell leukemia. In a typical embodiment, the patient has AML.

In another aspect, the invention features a method of treating a patient having a hematological malignancy, such as AML, by administering to the patient a composition containing an alpha4 antagonist, e.g., an anti-alpha4 antibody in a formulation suitable for IV or SC or IM administration. In one embodiment, the composition is administered as a regimen. In another embodiment, the method further includes selecting a patient suitable for treatment with the composition. A patient suitable for treatment, for example, has demonstrated a sign or symptom indicative of disease onset, such as a sign or symptom indicative of AML. A patient suitable for treatment may also express an elevated level of VLA4 protein on the surface of cells in a tissue sample (e.g., cells from a blood smear or bone marrow biopsy) as compared to the level of VLA4 protein expressed on cells in a human who does not have a hematological malignancy, such as AML.

In yet another embodiment, the method further includes administering to the patient a second therapeutic agent, such as, a chemotherapeutic agent, e.g., cytarabine (Ara-C), daunorubicin, idarubicin, etoposide, gemtuzumab ozogamicin, arsenic trioxide, or all-trans retinoic acid.

In another aspect, the invention features a method of evaluating a patient by determining if the patient meets a preselected criterion, and if the patient meets the preselected criterion approving, providing, prescribing, or administering an anti-alpha4 antibody formulation described herein to the patient. In one embodiment, the preselected criterion is the failure of the patient to adequately respond to a prior alternate therapeutic treatment or regimen, e.g., for treatment of a hematological malignancy, such as AML. In another embodiment, the preselected criterion is the absence of any signs or symptoms of progressive multifocal leukoencephalopathy (PML), or the absence of any diagnosis of PML. In another embodiment, the criterion is as described in PCT/US2007/075577 (published as WO/2008/021954), hereby incorporated by reference, which describes methods and systems for drug distribution.

In another aspect, the invention features a method of instructing a recipient on the administration of a formulation of natalizumab. The method includes instructing the recipient (e.g., an end user, patient, physician, retail or wholesale pharmacy, distributor, or pharmacy department at a hospital, nursing home clinic or HMO) that the drug should be administered to a patient subcutaneously or intramuscularly.

In another aspect, a method of distributing a composition described herein is provided. The composition contains natalizumab and is suitable for subcutaneous or intramuscular or intravenous administration. The method includes providing a recipient (e.g., an end user, patient, physician, retail or wholesale pharmacy, distributor, or pharmacy department at a hospital, nursing home clinic or HMO) with a package containing sufficient unit dosages of the drug to treat a patient for at least 6, 12, 24, or 36 months.

In another aspect, the invention features the use of a method or system described in PCT/US2007/075577 (published as WO/2008/021954) with a formulation described herein. Embodiments include a method of distributing a formulation described herein, monitoring or tracking the provision of a formulation described herein to a pharmacy, infusion center, or patient, monitoring one or more patients, selecting patients, or compiling or reporting data on the use of a formulation described herein. PCT/US2007/075577 (published as WO/2008/021954) is hereby incorporated by reference.

In another aspect, the invention features a method of selecting a patient for treatment with a composition containing an anti-alpha4 antibody. The method includes selecting or providing a patient who has a hematological malignancy, such as AML; and providing or administering a composition comprising an anti-alpha4 antibody, thereby treating the patient.

A “hematological malignancy” is a disorder, such as a cancer, that affects the blood, bone marrow, or lymph nodes. Hematological malignancies include leukemias, such as ALL, AML, CML, CLL, and HCL; lymphomas, such as Hodgkin's disease and Non-Hodgkin lymphoma; and multiple myeloma; myelodysplastic syndrome (MDS) (which can culminate in AML); a myeloproliferative disease, such as polycythemia vera (also called PV, PCV or polycythemia rubra vera (PRV)), Essential thrombocytosis (ET), myelofibrosis; and amyloid due to light-chain disease.

The term “treating” refers to administering a therapy in an amount, manner, and/or mode effective to improve a condition, symptom, or parameter associated with a disorder or to prevent progression of a disorder, to either a statistically significant degree or to a degree detectable to one skilled in the art. An effective amount, manner, or mode can vary depending on the subject and may be tailored to the subject.

An “anti-alpha4 antibody” refers to an antibody that binds to an alpha4 integrin, such as to the alpha4 subunit of the VLA-4 integrin, and at least partially inhibits an activity of the integrin. For example, an anti-alpha4 antibody may inhibit binding of the integrin to a cognate ligand, e.g., a cell surface protein such as VCAM-1, or to an extracellular matrix component, such as fibronectin or osteopontin. The effect of the inhibition may prevent an anti-alpha4 integrin from binding a cell, such as a bone marrow stromal cell. Alpha4 integrins are integrins whose alpha4 subunit associates with one or another of the beta subunits. Thus, the term “alpha4 integrin” refers to VLA-4, as well as integrins that contain beta1, beta7 or any other beta subunit (e.g., alpha4beta7, alpha4beta1). Thus, anti-alpha4 antibodies useful for treating a hematological malignancy include, for example, VLA-4 binding antibodies as well as alpha4beta7 antibodies, and antigen binding fragments thereof. An anti-alpha4 antibody may bind to alpha4 integrin with a K_{d} of less than about 10⁻⁶, 10⁻⁷, 10⁻⁸, 10⁻⁹, or 10⁻¹⁰ M.

A “VLA-4 binding antibody” refers to an antibody that can bind to a VLA-4 integrin, such as to the α4 subunit of the VLA-4 integrin, and at least partially inhibits an activity of a VLA-4, particularly a binding activity of a VLA-4 integrin or a signaling activity, e.g., ability to transduce a VLA-4 mediated signal. For example, a VLA-4 binding antibody may inhibit binding of VLA-4 to a cognate ligand of VLA-4, e.g., a cell surface protein such as VCAM-1, or to an extracellular matrix component, such as fibronectin or osteopontin. A VLA-4 binding antibody may bind to either the α4 subunit or the β1 subunit, or to both. In one embodiment, the antibody binds to the B1 epitope of α4. A VLA-4 binding antibody may bind to VLA-4 with a K_{d} of less than about 10⁻⁶, 10⁻⁷, 10⁻⁸, 10⁻⁹, or 10⁻¹⁰ M. VLA-4 is also known as alpha4/beta1 and CD29/CD49b. In one embodiment, the VLA-4 binding antibody is natalizumab, or has a K_{d} within 70%-130%, e.g., within 80%-125%, of the K_{d} of natalizumab.

As used herein, the term “antibody” refers to a protein that includes at least one immunoglobulin variable region, e.g., an amino acid sequence that provides an immunoglobulin variable domain or immunoglobulin variable domain sequence. For example, an antibody can include a heavy (H) chain variable region (abbreviated herein as VH), and a light (L) chain variable region (abbreviated herein as VL). In another example, an antibody includes two heavy (H) chain variable regions and two light (L) chain variable regions. The term “antibody” encompasses antigen-binding fragments of antibodies (e.g., single chain antibodies, Fab fragments, F(ab′)₂ fragments, Fd fragments, Fv fragments, and dAb fragments) as well as complete antibodies, e.g., intact immunoglobulins of types IgA, IgG, IgE, IgD, IgM (as well as subtypes thereof). The light chains of the immunoglobulin may be of types kappa or lambda. In one embodiment, the antibody is glycosylated. An antibody can be functional for antibody dependent cytotoxicity and/or complement-mediated cytotoxicity, or may be non-functional for one or both of these activities.

The VH and VL regions can be further subdivided into regions of hypervariability, termed “complementarity determining regions” (“CDR”), interspersed with regions that are more conserved, termed “framework regions” (FR). The extent of the FRs and CDRs has been precisely defined (see, Kabat, E. A., et al. (1991) Sequences of Proteins of Immunological Interest, Fifth Edition, U.S. Department of Health and Human Services, NIH Publication No. 91-3242; and Chothia, C. et al. (1987) J. Mol. Biol. 196:901-917). Kabat definitions are used herein. Each VH and VL is typically composed of three CDRs and four FRs, arranged from amino-terminus to carboxyl-terminus in the following order: FR1, CDR1, FR2, CDR2, FR3, CDR3, FR4.

An “immunoglobulin domain” refers to a domain from the variable or constant domain of immunoglobulin molecules. Immunoglobulin domains typically contain two β-sheets formed of about seven β-strands, and a conserved disulphide bond (see, e.g., A. F. Williams and A. N. Barclay 1988 Ann. Rev Immunol. 6:381-405).

As used herein, an “immunoglobulin variable domain sequence” refers to an amino acid sequence that can form the structure of an immunoglobulin variable domain. For example, the sequence may include all or part of the amino acid sequence of a naturally-occurring variable domain. For example, the sequence may omit one, two or more N- or C-terminal amino acids, internal amino acids, may include one or more insertions or additional terminal amino acids, or may include other alterations. In one embodiment, a polypeptide that includes an immunoglobulin variable domain sequence can associate with another immunoglobulin variable domain sequence to form a target binding structure (or “antigen binding site”), e.g., a structure that interacts with VLA-4.

The VH or VL chain of the antibody can further include all or part of a heavy or light chain constant region, to thereby form a heavy or light immunoglobulin chain, respectively. In one embodiment, the antibody is a tetramer of two heavy immunoglobulin chains and two light immunoglobulin chains. The heavy and light immunoglobulin chains can be connected by disulfide bonds. The heavy chain constant region typically includes three constant domains, CH1, CH2 and CH3. The light chain constant region typically includes a CL domain. The variable region of the heavy and light chains contains a binding domain that interacts with an antigen. The constant regions of the antibodies typically mediate the binding of the antibody to host tissues or factors, including various cells of the immune system (e.g., effector cells) and the first component (C1q) of the classical complement system.

One or more regions of an antibody can be human, effectively human, or humanized. For example, one or more of the variable regions can be human or effectively human. For example, one or more of the CDRs, e.g., HC CDR1, HC CDR2, HC CDR3, LC CDR1, LC CDR2, and LC CDR3, can be human (HC, heavy chain; LC, light chain). Each of the light chain CDRs can be human. HC CDR3 can be human. One or more of the framework regions can be human, e.g., FR1, FR2, FR3, and FR4 of the HC or LC. In one embodiment, all the framework regions are human, e.g., derived from a human somatic cell, e.g., a hematopoietic cell that produces immunoglobulins or a non-hematopoietic cell. In one embodiment, the human sequences are germline sequences, e.g., encoded by a germline nucleic acid. One or more of the constant regions can be human, effectively human, or humanized. In another embodiment, at least 70, 75, 80, 85, 90, 92, 95, or 98% of the framework regions (e.g., FR1, FR2, and FR3, collectively, or FR1, FR2, FR3, and FR4, collectively) or the entire antibody can be human, effectively human, or humanized. For example, FR1, FR2, and FR3 collectively can be at least 70, 75, 80, 85, 90, 92, 95, 98, or 99% identical to a human sequence encoded by a human germline segment.

An “effectively human” immunoglobulin variable region is an immunoglobulin variable region that includes a sufficient number of human framework amino acid positions such that the immunoglobulin variable region does not elicit an immunogenic response in a normal human. An “effectively human” antibody is an antibody that includes a sufficient number of human amino acid positions such that the antibody does not elicit an immunogenic response in a normal human.

A “humanized” immunoglobulin variable region is an immunoglobulin variable region that is modified such that the modified form elicits less of an immune response in a human than does the non-modified form, e.g., is modified to include a sufficient number of human framework amino acid positions such that the immunoglobulin variable region does not elicit an immunogenic response in a normal human. Descriptions of “humanized” immunoglobulins include, for example, U.S. Pat. No. 6,407,213 and U.S. Pat. No. 5,693,762. In some cases, humanized immunoglobulins can include a non-human amino acid at one or more framework amino acid positions.

All or part of an antibody can be encoded by an immunoglobulin gene or a segment thereof. Exemplary human immunoglobulin genes include the kappa, lambda, alpha (IgA1 and IgA2), gamma (IgG1, IgG2, IgG3, IgG4), delta, epsilon and mu constant region genes, as well as the myriad immunoglobulin variable region genes. Full-length immunoglobulin “light chains” (about 25 Kd or 214 amino acids) are encoded by a variable region gene at the NH₂-terminus (about 110 amino acids) and a kappa or lambda constant region gene at the COOH-terminus. Full-length immunoglobulin “heavy chains” (about 50 Kd or 446 amino acids), are similarly encoded by a variable region gene (about 116 amino acids) and one of the other aforementioned constant region genes, e.g., gamma (encoding about 330 amino acids).

The term “antigen-binding fragment” of a full length antibody refers to one or more fragments of a full-length antibody that retain the ability to specifically bind to a target of interest, e.g., VLA-4. Examples of binding fragments encompassed within the term “antigen-binding fragment” of a full length antibody include (i) a Fab fragment, a monovalent fragment consisting of the VL, VH, CL and CH1 domains; (ii) a F(ab′)₂ fragment, a bivalent fragment including two Fab fragments linked by a disulfide bridge at the hinge region; (iii) a Fd fragment consisting of the VH and CH1 domains; (iv) a Fv fragment consisting of the VL and VH domains of a single arm of an antibody, (v) a dAb fragment (Ward et al., (1989) Nature 341:544-546), which consists of a VH domain; and (vi) an isolated complementarity determining region (CDR) that retains functionality. Furthermore, although the two domains of the Fv fragment, VL and VH, are coded for by separate genes, they can be joined, using recombinant methods, by a synthetic linker that enables them to be made as a single protein chain in which the VL and VH regions pair to form monovalent molecules known as single chain Fv (scFv). See e.g., Bird et al. (1988) Science 242:423-426; and Huston et al. (1988) Proc. Natl. Acad. Sci. USA 85:5879-5883.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIGS. 1A, 1B and 1C are bar graphs depicting the amount of alpha4 and beta1 integrins on hematologic cell lines of acute myelogenous leukemia (AML) (FIG. 1A), multiple myeloma (MM) (FIG. 1B), and chronic lymphocytic leukemia (CLL) (FIG. 1C) as determined by flow cytometry.

FIG. 2 is a graph depicting binding of natalizumab to VLA-4 on tumor cell lines as measured by flow cytometry.

FIGS. 3A and 3B are graphs showing the effect of natalizumab on HL60 and KG1 AML tumor cell adhesion to VLA-4 ligands fibronectin (• FN), vascular adhesion molecule-1-Ig fusion protein (▪ VCAM-Ig), or bone marrow stromal cells (▴ BMSC), in the presence of natalizumab or an isotype control antibody in serial dilutions starting at 20 μg/ml. FIGS. 3C and 3D show inhibition of binding of HL60 and KG1 AML cells to VLA-4 ligands in the presence saturating levels of natalizumab (20 μg/mL) (solid bars) or isotype control (clear bars).

FIGS. 4A and 4B are graphs showing the effect of natalizumab on H929 and U266 mM tumor cell adhesion to VLA-4 ligands fibronectin (• FN), vascular adhesion molecule-1-Ig fusion protein (▪ VCAM-Ig), or bone marrow stromal cells (▴ BMSC). Adhesion was assayed in the presence of natalizumab or an isotype control antibody in serial dilutions starting at 20 μg/ml. FIG. 4C shows inhibition of binding of H929 mM cells to VLA-4 ligands in the presence saturating levels of natalizumab (20 μg/mL) (solid bars) or isotype control (clear bars).

FIGS. 5A and 5B are graphs showing the effect of natalizumab on Mec1 and JM1 CLL tumor cell adhesion to VLA-4 ligands fibronectin (• FN), vascular adhesion molecule-1-Ig fusion protein (▪ VCAM-Ig), or bone marrow stromal cells (▴ BMSC). Adhesion was assayed in the presence of natalizumab or an isotype control antibody in serial dilutions starting at 20 μg/ml. FIG. 5C shows inhibition of binding of Mec1 CLL cells to VLA-4 ligands in the presence saturating levels of natalizumab (20 μg/mL) (solid bars) or isotype control (clear bars).

FIGS. 6A-6D are graphs showing the results of natalizumab on cell adhesion mediated drug resistance. FIG. 6A depicts the percentage of viable HL60 cells remaining after the cells were cocultured for 24 hours with (▪) or without (□) BMSC, then exposed to the chemotherapy drug AraC (cytarabine) for 24 hours. FIG. 6B depicts the percentage of apoptotic cells (Annexin V+, 7AAD) after cocultured HL60 cells were incubated with natalizumab or isotype control antibody for 4 hr., and then exposed to an effective AraC dose as determined from FIG. 6A for 24 hours. FIGS. 6C and 6D show the results of similar experiments conducted with U266 cells exposed to melphalan.

FIG. 7 is a panel of Western blots assaying for P-STAT3, STAT3, P-JNK, JNK, P-MAPK, and MAPK levels in HL60, KG1 or U266 cells grown in suspension or cocultured with BMSCs and natalizumab, as indicated, for 30 minutes (HL60) or 4 hours (U266).

### DETAILED DESCRIPTION

The invention relates to treatments for, among other things, treating or preventing a hematological malignancy, such as AML. More particularly, provided herein are methods relating to the use of anti-alpha4 antibodies or antigen binding fragments thereof that are capable of blocking an interaction between an integrin containing an alpha4 subunit and a ligand for this integrin in the treatment of a hematological malignancy.

The VLA-4 (alpha4beta1) integrin is a cell-surface receptor for VCAM-1, fibronectin and possibly other molecules that bind with, or otherwise interact with, VLA-4. In this regard, such molecules that bind with, or otherwise interact with, an alpha4 subunit containing integrin are individually and collectively referred to as “alpha4 ligands.” The term VLA-4 (also called “a4β1,” “a4β1 integrin,” “alpha4beta1” and “alpha4beta1 integrin”) thus refers to polypeptides that are capable of binding to VCAM-1 and members of the extracellular matrix proteins, most particularly fibronectin, or homologs or fragments thereof, although it will be appreciated by workers of ordinary skill in the art that other ligands for VLA-4 may exist and can be analyzed using conventional methods.

It is known that the alpha4 subunit will associate with beta subunits other than beta1 so the term “alpha4 integrin” refers to those integrins whose alpha4 subunit associates with one or another of the beta subunits. A further example of an “alpha4” integrin is alpha4beta1. As used herein, the term “alpha4 integrin” refers to VLA-4, as well as integrins that contain beta1, beta7 or any other beta subunit.

The antagonists suitable for the methods described herein are not limited to a particular type or structure of molecule so that any agent capable of binding to any integrin containing an alpha4 subunit (e.g., VLA-4) on the surface of VLA4 bearing cells or alpha4beta7 integrin on the surface of alpha4beta7-bearing cells [see Lobb and Hemler, J. Clin. Invest., 94: 1722 1728 (1994)] or to their respective alpha4 ligands such as VCAM 1 and MadCAM, respectively, on the surface of VCAM-1 and MadCAM bearing cells, and which effectively blocks or coats VLA-4 (or alpha4beta7) or VCAM-1 (or MadCAM) (i.e., a “an alpha4 integrin binding agent” and “alpha4 integrin ligand binding agent,” respectively), is considered to be an equivalent of the antagonists described herein.

An integrin “antagonist” (also referred to herein as an “alpha4 antagonist”) includes any compound that inhibits an alpha4 integrins from binding with an alpha4 integrin ligand and/or receptor. Anti-integrin antibody-containing proteins as well as other molecules, such as soluble forms of the ligand proteins for integrins are useful. Soluble forms of the ligand proteins for alpha4 integrins include soluble VCAM-1 or collagen peptides, VCAM-1 fusion proteins, or bifunctional VCAM-1/Ig fusion proteins. For example, a soluble form of an alpha4 integrin ligand or a fragment thereof may be administered to bind to integrin, and in some instances, compete for an integrin binding site on cells, thereby leading to effects similar to the administration of antagonists such as anti-alpha4 integrin (e.g., alpha4 beta7 antibodies or VLA-4 antibodies). In particular, soluble alpha4 integrin mutants that bind alpha 4 integrin ligand but do not elicit integrin-dependent signaling are suitable for use in the described methods. Such mutants can act as competitive inhibitors of wild type integrin protein and are considered “antagonists.” Other suitable antagonists are “small molecules,” as defined below.

Agents that antagonize the action of more than one alpha4 integrin, such as a single small molecule or antibody, or antibody fragment, that antagonizes several alpha4 integrins, e.g., VLA-4 and alpha4 beta 7, or other combinations of alpha4 integrins are suitable for treating hematological malignancies. Combinations of different molecules, such that the combined activity antagonizes the action of more than one alpha4 integrin, are also suitable for the methods described herein.

In some embodiments, certain integrin antagonists are fused or otherwise conjugated to, for instance, an antibody or antibody fragment, e.g., an immunoglobulin or fragment thereof, and are not limited to a particular types or structures of an integrin or ligand or other molecule. Thus, any agent capable of forming a fusion protein and capable of binding to alpha4 integrin ligands, and which effectively blocks or coats alpha4beta7 or VLA-4 integrin, is considered to be an equivalent of the antagonists used in the examples herein.

An “antagonist of the alpha4 integrin ligand/alpha4 integrin interaction” refers to an agent, e.g., a polypeptide or other molecule, which can inhibit or block alpha4 ligand (e.g., VCAM-1) or alpha4 integrin (e.g., alpha4beta7 or VLA-4)-mediated binding, or which can otherwise modulate alpha4 ligand or alpha4 integrin function, such as by inhibiting or blocking alpha4-ligand mediated alpha4 integrin signal transduction or alpha4 ligand-mediated alpha4 ligand signal transduction and which is effective in the treatment of a hematological malignancy, such as AML, in the same manner as are anti-alpha4 integrin antibodies.

An antagonist of the VCAM-1/VLA-4 interaction is an agent that has one or more of the following properties: (1) it coats, or binds to, VLA-4 on the surface of a VLA-4 bearing cell (e.g., an AML cell) with sufficient specificity to inhibit a VLA-4-ligand/VLA-4 interaction, e.g., the VCAM-1/VLA-4 interaction between bone stromal cells and myeloma cells; (2) it coats, or binds to, VLA-4 on the surface of a VLA-4 bearing cell (i.e., a myeloma cell) with sufficient specificity to modify, e.g., to inhibit, transduction of a VLA-4-mediated signal, e.g., VLA-4/VCAM-1-mediated signaling; (3) it coats, or binds to, a VLA-4 ligand, (e.g., VCAM1) on bone stromal cells with sufficient specificity to inhibit the VLA-4/VCAM interaction; (4) it coats, or binds to, a VLA-4-ligand (e.g., VCAM-1) on bone stromal cells with sufficient specificity to modify, e.g., to inhibit, transduction of VLA-4-ligand mediated VLA-4 signaling, e.g., VCAM-1-mediated VLA-4 signaling. In some embodiments, the antagonist has one or both of properties 1 and 2. In other embodiments the antagonist has one or both of properties 3 and 4. Moreover, more than one antagonist can be administered to a patient, e.g., an agent that binds to VLA-4 can be combined with an agent that binds to VCAM-1.

For example, antibodies or antibody fragments as well as soluble forms of the natural binding proteins for VLA-4 and VCAM-1 are useful. Soluble forms of the natural binding proteins for VLA-4 include soluble VCAM-1 peptides, VCAM-1 fusion proteins, bifunctional VCAM-1/Ig fusion proteins, fibronectin, fibronectin having an alternatively spliced non-type m connecting segment, and fibronectin peptides containing the amino acid sequence EILDV or a similar conservatively substituted amino acid sequence. Soluble forms of the natural binding proteins for VCAM-1 include soluble VLA-4 peptides, VLAD fusion proteins, bifunctional VLA-4/Ig fusion proteins and the like. As used herein, a “soluble VLA-4 peptide” or a “soluble VCAM-1 peptide” is a VLA-4 or VCAM-1 polypeptide incapable of anchoring itself in a membrane. Such soluble polypeptides include, for example, VLA-4 and VCAM polypeptides that lack a sufficient portion of their membrane spanning domain to anchor the polypeptide or are modified such that the membrane spanning domain is non-functional. These binding agents can act by competing with the cell-surface binding protein for VLA-4 or by otherwise altering VLA-4 function. For example, a soluble form of VCAM-1 (see, e.g., Osborn et al. 1989, Cell, 59: 1203 1211) or a fragment thereof may be administered to bind to VLA-4, such as to compete for a VLA-4 binding site on myeloma cells, thereby leading to effects similar to the administration of antagonists, such as small molecules or anti-VLA-4 antibodies.

In another example, VCAM-1, or a fragment thereof which is capable of binding to VLA-4 on the surface of VLA-4 bearing myeloma cells, e.g., a fragment containing the two N-terminal domains of VCAM-1, can be fused to a second peptide, e.g., a peptide which increases the solubility or the in vivo life time of the VCAM-1 moiety. The second peptide can be a fragment of a soluble peptide, such as a human peptide or a plasma protein, or a member of the immunoglobulin superfamily. Typically, the second peptide is IgG or a portion or fragment thereof, e.g., the human IgG1 heavy chain constant region and includes, at least the hinge, CH2 and CH3 domains.

Agents that mimic the action of peptides (e.g., organic molecules called “small molecules”) capable of disrupting the alpha4 integrin/alpha4 integrin ligand interaction by, for instance, blocking VLA-4 by binding VLA-4 receptors on the surface of cells or blocking VCAM-1 by binding VCAM-1 receptors on the surface of cells. These “small molecules” may themselves be small peptides, or larger peptide-containing organic compounds or non-peptidic organic compounds. A “small molecule” is not intended to encompass an antibody or antibody fragment. Although the molecular weight of such “small” molecules is generally less than 2000, this figure is not intended as an absolute upper limit on molecular weight.

For instance, small molecules such as oligosaccharides that mimic the binding domain of a VLA-4 ligand and fit the receptor domain of VLA-4 may be employed. (See, J. J. Devlin et al., 1990, Science 249: 400-406 (1990), J. K. Scott and G. P. Smith, 1990, Science 249: 386 390, and U.S. Pat. No. 4,833,092 (Geysen), all incorporated herein by reference. Conversely, small molecules that mimic the binding domain of a VCAM-1 ligand and fit the receptor domain of VCAM-1 may be employed.

Small molecules described in WO 06/131200 and in US2007/0004775, both of which are incorporated herein by reference, are also suitable for use in treatment of hematological malignancies.

Examples of other small molecules useful in the invention can be found in Komoriya et al. (“The Minimal Essential Sequence for a Major Cell Type-Specific Adhesion Site (CS1) Within the Alternatively Spliced Type III Connecting Segment Domain of Fibronectin Is Leucine-Aspartic Acid-Valine”, J. Biol. Chem., 266 (23), pp. 15075 79 (1991)). They identified the minimum active amino acid sequence necessary to bind VLA-4 and synthesized a variety of overlapping peptides based on the amino acid sequence of the CS-1 region (the VLA-4 binding domain) of a particular species of fibronectin. They identified an 8-amino acid peptide, Glu-Ile-Leu-Asp-Val-Pro-Ser-Thr, as well as two smaller overlapping pentapeptides, Glu-Ile-Leu-Asp-Val and Leu-Asp-Val-Pro-Ser, that possessed inhibitory activity against fibronectin-dependent cell adhesion. Certain larger peptides containing the LDV sequence were subsequently shown to be active in vivo (T. A. Ferguson et al., “Two Integrin Binding Peptides Abrogate T-cell-Mediated Immune Responses In Vivo”, Proc. Natl. Acad. Sci. USA, 88, pp. 8072 76 (1991); and S. M. Wahl et al., “Synthetic Fibronectin Peptides Suppress Arthritis in Rats by Interrupting Leukocyte Adhesion and Recruitment”, J. Clin. Invest., 94, pp. 655 62 (1994)). A cyclic pentapeptide, Arg-Cys-Asp-TPro-Cys (wherein TPro denotes 4-thioproline), which can inhibit both VLA-4 and VLA-5 adhesion to fibronectin has also been described. (See, e.g., D. M. Nowlin et al. “A Novel Cyclic Pentapeptide Inhibits Alpha4Beta1 Integrin-mediated Cell Adhesion”, J. Biol. Chem., 268(27), pp. 20352 59 (1993); and PCT publication PCT/US91/04862).

Examples of other small molecule VLAW inhibitors have been reported, for example, in Adams et al. “Cell Adhesion Inhibitors”, PCT US97/13013, describing linear peptidyl compounds containing beta-amino acids which have cell adhesion inhibitory activity. International patent applications WO 94/15958 and WO 92/00995 describe cyclic peptide and peptidomimetic compounds with cell adhesion inhibitory activity. International patent applications WO 93/08823 and WO 92108464 describe guanidinyl-, urea- and thiourea-containing cell adhesion inhibitory compounds. U.S. Pat. No. 5,260,277 describes guanidinyl cell adhesion modulation compounds.

Such small molecules mimetic agents may be produced by synthesizing a plurality of peptides semi-peptidic compounds or non-peptidic, organic compounds, and then screening those compounds for their ability to inhibit the alpha4 integrin/alpha4 integrin ligand interaction. See generally U.S. Pat. No. 4,833,092, Scott and Smith, “Searching for Peptide Ligands with an Epitope Library”, Science, 249, pp. 386 90 (1990), and Devlin et al., “Random Peptide Libraries: A Source of Specific Protein Binding Molecules”, Science, 249, pp. 40407 (1990).

In other embodiments, an agent that is used to bind to, including block or coat, cell-surface alpha4 integrin and/or alpha4 integrin ligand is an anti-VLA-4 and/or anti-alpha4beta7 monoclonal antibody or antibody fragment. Antibodies and antibody fragments for treatment, in particular for human treatment, include human, humanized, and chimeric antibodies and antibody fragments, Fab, Fab′, F(ab′)₂ and F(v) antibody fragments, and monomers or dimers of antibody heavy or light chains or mixtures thereof. Typically, the binding agent is a monoclonal antibody that binds VLA-4.

Hematological Malignancies

Methods are provided for treating a patient having a hematological disorder with a composition containing a VLA-4 binding antibody. Hematological malignancies are disorders, such as a cancer, that affect the blood, bone marrow, and/or lymph nodes. Hematological malignancies include leukemias, such as ALL, AML, CML, CLL, and HCL; lymphomas, such as Hodgkin's disease and Non-Hodgkin lymphoma; and multiple myeloma; myelodysplastic syndrome (MDS) (which can culminate in AML); a myeloproliferative disease, such as polycythemia vera (also called PV, PCV or polycythemia rubra vera (PRV)), Essential thrombocytosis (ET), myelofibrosis; and amyloid due to light-chain disease.

Patients having a hematological malignancy may be identified by analysis of blood count and blood film by, for example, light microscopy, which is useful for identifying malignant cells. A biopsy, such as from bone marrow, can also be used to identify malignant cells, and a biopsy from a lymph node can be useful for identifying a lymphadenopathy.

Acute Myelogenous Leukemia (AML)

A VLA-4 binding antibody is useful for the treatment of a leukemia, such as AML. Leukemias are cancers that originate in the bone marrow, where the malignant cells are white blood cells (leukocytes). Acute myelogenous leukemia (also called acute myelocytic leukemia, acute myeloblastic leukemia, acute granulocytic leukemia, and acute nonlymphocytic leukemia) is a malignancy that arises in either granulocytes or monocytes. AML is characterized by the uncontrolled, exaggerated growth and accumulation of cells called leukemic blasts, which fail to function as normal blood cells, and the blockade of the production of normal marrow cells, leading to a deficiency of red cells (anemia), and platelets (thrombocytopenia) and normal white cells (especially neutrophils, i.e., neutropenia) in the blood.

All subtypes of AML are suitable for treatment with a VLA-4 binding antibody. The subtypes of AML are classified based on the stage of development myeloblasts have reached at the time of diagnosis. The categories and subsets allow the physician to decide what treatment works best for the cell type and how quickly the disease may develop. The subsets are: M0, myeloblastic, on special analysis; M1, Myeloblastic, without maturation; M2, Myeloblastic, with maturation; M3, Promyelocytic; M4, Myelomonocytic; M5, Monocytic; M6, Erythroleukemia; and M7, Megakaryocytic. A VLA-4 antibody can be administered with a secondary agent that is particularly suited to the subtype of AML. For example, acute promyelocytic leukemia (APL) and acute monocytic leukemia are subtypes of AML that need different treatment than other subtypes of AML. A second agent for treatment of APL can include all-trans retinoic acid (ATRA) or an antimetabolite, such as cytarabine. A second agent for treatment of acute monocytic leukemia can include a deoxyadenosine analog, such as 2-chloro-2′-deoxyadenosine (2-CDA).

Risk factors of AML include the presence of certain genetic disorders, such as Down syndrome, Fanconi anemia, Shwachman-Diamond syndrome and others. A patient having AML and a genetic disorder can be administered a VLA-4 binding antibody and a second agent to treat a symptom of the genetic disorder. For example, a patient with AML and Fanconi anemia can be administered a VLA-4 binding antibody and an antibiotic.

Other risk factors for AML include chemotherapy or radiotherapy for treatment of a different cancer, tobacco smoke, and exposure to large amounts of benzene.

Therapy can be deemed to be effective if there is a statistically significant difference in the rate or proportion of malignant cells in the blood stream or bone marrow. Therapy is deemed to be effective, for example, when remission is achieved, which is when there are no signs of malignant cells.

Efficacy of administering a first agent and, optionally, a second agent, can also be evaluated based on, for example, the decrease of number of malignant cells found in the blood stream, a decrease in frequency or severity of bacterial or viral infection, increased rate of wound healing, and the general feeling of the patient, including increased energy level and decreased soreness in bones and joints.

In addition to, or prior to human studies, an animal model can be used to evaluate the efficacy of using the two agents. For example, mice can be administered a first and second agent described herein, and then the mice are evaluated for characteristic criteria to determine the efficacy of using the two agents in the model. Such models are known in the art, e.g., See Drug Discovery Today: Disease Models 3(2): 137-142 (2006); Blood, online Mar. 30, 2009; DOI 10.1182/blood-2009-01-198937; and on the worldwide web at emice.nci.nih.gov/emice/mouse_models/organ_models/hema_models/hema_mouse_tools.

Natalizumab and Other VLA-4 Binding Antibodies

Antibodies suitable for use in treatment of a hematological malignancy, such as AML, include natalizumab, an α4 integrin binding antibody. Natalizumab (USAN name) has the antibody code number AN100226, and is also called “TYSABRI™” The amino acid sequence of the light chain and heavy chain of natalizumab prior to any in vivo modifications (e.g., clipping of amino acids) is shown in Table 1-1 and Table 1-2.

TABLE 1-1
Sequence of Natalizumab Light Chain

(SEQ ID NO: 1)

| | | | | | |
|---|---|---|---|---|---|
| | 10 | 20 | 30 | 40 | 50 |
| 1 | DIQMTQSPSS | LSASVGDRVT | ITCKTSQDIN | KYMAWYQQTP | GKAPRLLIHY |
| 51 | TSALQPGIPS | RFSGSGSGRD | YTFTISSLQP | EDIATYYCLQ | YDNLWTFGQG |
| 101 | TKVEIKRTVA | APSVFIFPPS | DEQLKSGTAS | VVCLLNNFYP | REAKVQWKVD |
| 151 | NALQSGNSQE | SVTEQDSKDS | TYSLSSTLTL | SKADYEKHKV | YACEVTHQGL |
| 201 | SSPVTKSFNR | GEC | | | |

TABLE 1-2
Sequence of Natalizumab Heavy Chain

(SEQ ID NO: 2)

| | | | | |
|---|---|---|---|---|
| 10 | 20 | 30 | 40 | 50 |
| Q¹VQLVQSGAE | VKKPGASVKV | SCKASGFNIK | DTYIHWVRQA | PGQRLEWMGR |
| IDPANGYTKY | DPKFQGRVTI | TADTSASTAY | MELSSLRSED | TAVYYCAREG |
| YYGNYGVYAM | DYWGQGTLVT | VSSASTKGPS | VFPLAPCSRS | TSESTAALGC |
| LVKDYFPEPV | TVSWNSGALT | SGVHTFPAVL | QSSGLYSLSS | VVTVPSSSLG |
| TKTYTCNVDH | KPSNTKVDKR | VESKYGPPCP | SCPAPEFLGG | PSVFLFPPKP |
| KDTLMISRTP | EVTCVVVDVS | QEDPEVQFNW | YVDGVEVHNA | KTKPREEQFN |
| STYRVVSVLT | VLHQDWLNGK | EYKCKVSNKG | LPSSIEKTIS | KAKGQPREPQ |
| VYTLPPSQEE | MTKNQVSLTC | LVKGFYPSDI | AVEWESNGQP | ENNYKTTPPV |
| LDSDGSFFLY | SRLTVDKSRW | QEGNVFSCSV | MHEALHNHYT | QKSLSLSLGK² |

¹Glutamine cyclized to pyroGlutamic Acid
²Lysine is removed posttranslationally

Natalizumab inhibits the migration of leukocytes from the blood to the central nervous system. Natalizumab binds to VLA-4 (also called α4β1) on the surface of activated T-cells and other mononuclear leukocytes. It can disrupt adhesion between the T-cell and endothelial cells, and thus prevent migration of mononuclear leukocytes across the endothelium and into the parenchyma. As a result, the levels of proinflammatory cytokines can also be reduced.

Natalizumab and related VLA-4 binding antibodies are described, e.g., in U.S. Pat. No. 5,840,299. Monoclonal antibodies 21.6 and HP1/2 are exemplary murine monoclonal antibodies that bind VLA-4. Natalizumab is a humanized version of murine monoclonal antibody 21.6 (see, e.g., U.S. Pat. No. 5,840,299). A humanized version of HP1/2 has also been described (see, e.g., U.S. Pat. No. 6,602,503). Several additional VLA-4 binding monoclonal antibodies, such as HP2/1, HP2/4, L25 and P4C2, are described, e.g., in U.S. Pat. No. 6,602,503; Sanchez-Madrid et al., 1986 Eur. J. Immunol., 16:1343-1349; Hemler et al., 1987 J. Biol. Chem. 2:11478-11485; Issekutz and Wykretowicz, 1991, J. Immunol., 147: 109 (TA-2 mab); Pulido et al., 1991 J. Biol. Chem., 266:10241-10245; and U.S. Pat. No. 5,888,507).

Some VLA-4 binding antibodies recognize epitopes of the α4 subunit that are involved in binding to a cognate ligand, e.g., VCAM-1 or fibronectin. Many such antibodies inhibit binding of VLA-4 to cognate ligands (e.g., VCAM-1 and fibronectin).

Some useful VLA-4 binding antibodies can interact with VLA-4 on cells, e.g., lymphocytes, but do not cause cell aggregation. However, other VLA-4 binding antibodies have been observed to cause such aggregation. HP1/2 does not cause cell aggregation. The HP1/2 monoclonal antibody (Sanchez-Madrid et al., 1986) has an extremely high potency, blocks VLA-4 interaction with both VCAM1 and fibronectin, and has the specificity for epitope B on VLA-4. This antibody and other B epitope-specific antibodies (such as B1 or B2 epitope binding antibodies; Pulido et al., 1991, supra) represent one class of VLA-4 binding antibodies that can be used in the formulations and methods described herein.

An exemplary VLA-4 binding antibody has one or more CDRs, e.g., all three HC CDRs and/or all three LC CDRs of a particular antibody disclosed herein, or CDRs that are, in sum, at least 80, 85, 90, 92, 94, 95, 96, 97, 98, 99% identical to such an antibody, e.g., natalizumab. In one embodiment, the H1 and H2 hypervariable loops have the same canonical structure as those of an antibody described herein. In one embodiment, the L1 and L2 hypervariable loops have the same canonical structure as those of an antibody described herein.

In one embodiment, the amino acid sequence of the HC and/or LC variable domain sequence is at least 70, 80, 85, 90, 92, 95, 97, 98, 99, or 100% identical to the amino acid sequence of the HC and/or LC variable domain of an antibody described herein, e.g., natalizumab. The amino acid sequence of the HC and/or LC variable domain sequence can differ by at least one amino acid, but no more than ten, eight, six, five, four, three, or two amino acids from the corresponding sequence of an antibody described herein, e.g., natalizumab. For example, the differences may be primarily or entirely in the framework regions.

The amino acid sequences of the HC and LC variable domain sequences can be encoded by a nucleic acid sequence that hybridizes under high stringency conditions to a nucleic acid sequence described herein or one that encodes a variable domain or an amino acid sequence described herein. In one embodiment, the amino acid sequences of one or more framework regions (e.g., FR1, FR2, FR3, and/or FR4) of the HC and/or LC variable domain are at least 70, 80, 85, 90, 92, 95, 97, 98, 99, or 100% identical to corresponding framework regions of the HC and LC variable domains of an antibody described herein. In one embodiment, one or more heavy or light chain framework regions (e.g., HC FR1, FR2, and FR3) are at least 70, 80, 85, 90, 95, 96, 97, 98, or 100% identical to the sequence of corresponding framework regions from a human germline antibody.

Calculations of “homology” or “sequence identity” between two sequences (the terms are used interchangeably herein) are performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). The optimal alignment is determined as the best score using the GAP program in the GCG software package with a Blossum 62 scoring matrix with a gap penalty of 12, a gap extend penalty of 4, and a frameshift gap penalty of 5. The amino acid residues or nucleotides at corresponding amino acid positions or nucleotide positions are then compared. When a position in the first sequence is occupied by the same amino acid residue or nucleotide as the corresponding position in the second sequence, then the molecules are identical at that position (as used herein amino acid or nucleic acid “identity” is equivalent to amino acid or nucleic acid “homology”). The percent identity between the two sequences is a function of the number of identical positions shared by the sequences.

As used herein, the term “hybridizes under high stringency conditions” describes conditions for hybridization and washing. Guidance for performing hybridization reactions can be found in Current Protocols in Molecular Biology, John Wiley & Sons, N.Y. (1989), 6.3.1-6.3.6, which is incorporated by reference. Aqueous and nonaqueous methods are described in that reference and either can be used. High stringency hybridization conditions include hybridization in 6×SSC at about 45° C., followed by one or more washes in 0.2×SSC, 0.1% SDS at 65° C., or substantially similar conditions.

Exemplary Second Agents

In some cases, a method of treating a hematological disorder includes administering a VLA-4 binding antibody and a second therapeutic agent.

In one implementation, the VLA-4 binding antibody and second agent is provided as a co-formulation, and the co-formulation is administered to the subject. It is further possible, e.g., at least 24 hours before or after administering the co-formulation, to administer separately one dose of the antibody formulation and then one dose of a formulation containing the second agent. In another implementation, the antibody and the second agent are provided as separate formulations, and the step of administering includes sequentially administering the antibody and the second agent. The sequential administrations can be provided on the same day (e.g., within one hour of one another or at least 3, 6, or 12 hours apart) or on different days.

In one embodiment, the antibody and the second agent are each administered as a plurality of doses separated in time. The antibody and the second agent are generally each administered according to a regimen. The regimen for one or both may have a regular periodicity. The regimen for the antibody can have a different periodicity from the regimen for the second agent, e.g., one can be administered more frequently than the other. In one implementation, one of the antibody and the second agent is administered once weekly and the other once monthly. In another implementation, one of the antibody and the second agent is administered continuously, e.g., over a period of more than 30 minutes but less than 1, 2, 4, or 12 hours, and the other is administered as a bolus. The antibody and the second agent can be administered by any appropriate method, e.g., subcutaneously, intramuscularly, or intravenously.

In some embodiments, each of the antibody and the second agent is administered at the same dose as each is prescribed for monotherapy. In other embodiments, the antibody is administered at a dosage that is equal to or less than an amount required for efficacy if administered alone. Likewise, the second agent can be administered at a dosage that is equal to or less than an amount required for efficacy if administered alone.

Non-limiting examples of second agents for treating a hematological malignancy, such as AML in combination with a VLA-4 binding antibody include cytarabine (also called AraC or cytosine arabinoside), daunorubicin (Daunomycin), doxorubicin, temozolomide, daunomycin, dactinomycin, epirubicin, idarubicin, esorubicin, bleomycin, mafosfamide, ifosfamide, gemtuzumab ozogamicin, rituximab, ofatumumab, tositumomab, ibritumomab tiuxetan, epratuzumab, alemtuzumab, fludarabine, bis-chloroethylnitrosurea, busulfan, mitomycin C, actinomycin D, mithramycin, prednisone, hydroxyprogesterone, testosterone, tamoxifen, dacarbazine, procarbazine, hexamethylmelamine, pentamethylmelamine, mitoxantrone, amsacrine, chlorambucil, methylcyclohexylnitrosurea, nitrogen mustards, melphalan, cyclophosphamide, 6-mercaptopurine, 6-thioguanine, 5-azacytidine, hydroxyurea, deoxycoformycin (pentostatin), 2-chlorodeoxyadenosine (cladribine), 4-hydroxyperoxycyclophosphor-amide, 5-fluorouracil (5-FU), 5-fluorodeoxyuridine (5-FUdR), melphalan, methotrexate (MTX), colchicine, taxol, vincristine, vinblastine, etoposide (VP-16), trimetrexate, irinotecan, topotecan, gemcitabine, teniposide, cisplatin, carboplatin, and diethylstilbestrol (DES). See, generally, The Merck Manual of Diagnosis and Therapy, 15th Ed. 1987, pp. 1206-1228, Berkow et al., eds., Rahway, N.J. When used with the dsRNAs featured in the invention, such chemotherapeutic agents may be used individually (e.g., 5-FU and oligonucleotide), sequentially (e.g., 5-FU and oligonucleotide for a period of time followed by MTX and oligonucleotide), or in combination with one or more other such chemotherapeutic agents (e.g., 5-FU, MTX and oligonucleotide, or 5-FU, radiotherapy and oligonucleotide). Anti-inflammatory drugs, including but not limited to nonsteroidal anti-inflammatory drugs and corticosteroids, and antiviral drugs, including but not limited to ribavirin, vidarabine, acyclovir and ganciclovir, may also be combined in compositions featured in the invention. See, generally, The Merck Manual of Diagnosis and Therapy, 15th Ed., Berkow et al., eds., 1987, Rahway, N.J., pages 2499-2506 and 46-49, respectively).

A second therapeutic agent can also be a proteasome inhibitor, such as bortezomib; a Flt3 inhibitor, such as sorafenib; or a stem cell mobilizing agent, such as plerixafor.

Other non-RNAi chemotherapeutic agents are also within the scope of this invention.

Two or more combined compounds may be used together or sequentially.

In some embodiments, a patient having a hematological malignancy is administered a therapy in addition to the administration of the VLA-4 binding antibody. For example, the patient is administered a blood transfusion, radiotherapy, immunotherapy or a bone marrow transplant. In one embodiment, the patient has AML, and the patient receives a blood stem cell transplant, in addition to a VLA-4 antibody treatment.

In some embodiments, a second agent may be used to treat one or more symptoms or side effects of the malignancy. Side effects include, for example, anemia (which may cause fatigue and shortness of breath), increased infections, pain in the bones and joints, mild fever, bruising or bleeding more easily (e.g., bleeding gums or nose, or cuts that heal slowly). Such agents include, e.g., antibiotics or iron supplements. Exemplary antibiotics include, e.g., aclacinomycins, actinomycin, authramycin, azaserine, bleomycins, cactinomycin, calicheamicin, carabicin, caminomycin, carzinophilin, chromomycins, dactinomycin, daunorubicin, detorubicin, 6-diazo-5-oxo-L-norleucine, doxorubicin, epirubicin, esorubicin, idarubicin, marcellomycin, mitomycins, mycophenolic acid, nogalamycin, olivomycins, peplomycin, potfiromycin, puromycin, quelamycin, rodorubicin, streptonigrin, streptozocin, tubercidin, ubenimex, zinostatin, zorubicin; anti-metabolites such as methotrexate and 5-fluorouracil (5-FU); folic acid analogues such as denopterin, methotrexate, pteropterin, trimetrexate; purine analogs such as fludarabine, 6-mercaptopurine, thiamiprine, thioguanine; pyrimidine analogs such as ancitabine, azacitidine, 6-azauridine, carmofur, cytarabine, dideoxyuridine, doxifluridine, enocitabine, floxuridine, androgens such as calusterone, dromostanolone propionate, epitiostanol, mepitiostane, testolactone; anti-adrenals such as aminoglutethimide, mitotane, trilostane; folic acid replenisher such as frolinic acid; aceglatone; aldophosphamide glycoside; aminolevulinic acid; amsacrine; bestrabucil; bisantrene; edatraxate; defofamine; demecolcine; diaziquone; duocarmycin, maytansin, auristatin, elfomithine; elliptinium acetate; etoglucid; gallium nitrate; hydroxyurea; lentinan; lonidamine; mitoguazone; mitoxantrone (Novantrone); mopidamol; nitracrine; pentostatin; phenamet; pirarubicin; podophyllinic acid; 2-ethylhydrazide; procarbazine; PSK™; razoxane; sizofuran; spirogermanium; tenuazonic acid; triaziquone; 2,2′,2″-trichlorotriethyla-mine; urethan; vindesine; dacarbazine; mannomustine; mitobronitol; mitolactol; pipobroman; gacytosine; arabinoside (also called “Ara-C,” cytarabine and cytosine arabinoside); cyclophosphamide; thiotepa; taxanes, e.g. paclitaxel (TAXOL™, Bristol-Myers Squibb Oncology, Princeton, N.J.) and docetaxel (TAXOTERE™, Rhone-Poulenc Rorer, Antony, France); chlorambucil; gemcitabine; 6-thioguanine; mercaptopurine; methotrexate; platinum analogs such as cisplatin and carboplatin; vinblastine; platinum; etoposide (VP-16); ifosfamide; mitomycin C; mitoxantrone (Novantrone); vincristine; vinorelbine (Navelbine); novantrone; teniposide; daunorubicin (Daunomycin); aminopterin; capecitabine (Xeloda); ibandronate; camptothecin-11 (CPT-1); topoisomerase inhibitor RFS 2000; difluoromethylornithine (DMFO); retinoic acid; esperamicins; capecitabine; and pharmaceutically acceptable salts, acids or derivatives of any of the above. Also included as suitable chemotherapeutic cell conditioners are anti-hormonal agents that act to regulate or inhibit hormone action on tumors such as anti-estrogens including for example tamoxifen, raloxifene, aromatase inhibiting 4(5)-imidazoles, 4-hydroxytamoxifen, trioxifene, keoxifene, LY 117018, onapristone, and toremifene (Fareston); and anti-androgens such as flutamide, nilutamide, bicalutamide, leuprolide, goserelin, doxorubicin, daunorubicin, duocarmycin, vincristin, and vinblastin.

In some embodiments, the second agent is a second anti-alpha4 binding antibody, or a bispecific antibody. For example, a VLA-4 binding antibody and an alpha4beta7 binding antibody (or fragments thereof) can be administered for the treatment of a hematological malignancy.

In addition to a second agent, it is also possible to deliver still other agents to the subject. However, in some embodiments, no protein or no biologic, other than the VLA-4 binding antibody and second agent, are administered to the subject as a pharmaceutical composition. The VLA-4 binding antibody and the second agent may be the only agents that are delivered by injection. In embodiments in which the VLA-4 binding antibody and the second agent are recombinant proteins, the VLA-4 binding antibody and second agent may be the only recombinant agents administered to the subject, or at least the only recombinant agents that modulate immune or inflammatory responses. In still other embodiments, the VLA-4 binding antibody alone is the only recombinant agent or the only biologic administered to the subject.

Pharmaceutical Compositions

The compositions described herein are formulated as pharmaceutical compositions. Typically, a pharmaceutical composition includes a pharmaceutically acceptable carrier. As used herein, “pharmaceutically acceptable carrier” includes any and all solvents, dispersion media, coatings, antibacterial and antifungal agents, isotonic and absorption delaying agents, and the like that are physiologically compatible.

A “pharmaceutically acceptable salt” refers to a salt that retains the desired biological activity of the antibody and does not impart any undesired toxicological effects (see e.g., Berge, S. M., et al. (1977) J. Pharm. Sci. 66:1-19). Examples of such salts include acid addition salts and base addition salts. Acid addition salts include those derived from nontoxic inorganic acids, such as hydrochloric, nitric, phosphoric, sulfuric, hydrobromic, hydroiodic, and the like, as well as from nontoxic organic acids such as aliphatic mono- and dicarboxylic acids, phenyl-substituted alkanoic acids, hydroxy alkanoic acids, aromatic acids, aliphatic and aromatic sulfonic acids, free amino acids, and the like. Base addition salts include those derived from alkaline earth metals, such as sodium, potassium, magnesium, calcium and the like, as well as from nontoxic organic amines, such as N,N′-dibenzylethylenediamine, N-methylglucamine, chloroprocaine, choline, diethanolamine, ethylenediamine, procaine and the like.

Typically physiologically compatible agents, such as free amino acids, the hydrochloride salts, sodium salts, or potassium salts of free amino acids are used as excipients in pharmaceutical formulations to promote stability of the antibody. The formulations herein can include additives such as glycerol, mannitol, sorbitol, and other polyols, as well as sugars (e.g., sucrose), to promote stability.

The pharmaceutical compositions containing VLA-4 binding antibodies can be in the form of a liquid solution (e.g., injectable and infusible solutions). Such compositions can be administered by a parenteral mode (e.g., subcutaneous, intraperitoneal, or intramuscular injection). The phrases “parenteral administration” and “administered parenterally” as used herein mean modes of administration other than enteral and topical administration, usually by injection, and include, subcutaneous or intramuscular administration, as well as intravenous, intracapsular, intraorbital, intracardiac, intradermal, intraperitoneal, transtracheal, subcuticular, subcapsular, subarachnoid, intraspinal, epidural, intrahepatic, intrarticular, intrasynovial, intrathecal, intralesional, intralymphatic, intracranial and intrasternal injection and infusion. In some embodiments, a substance such as hyaluronidase may be administered before the antibody to allow larger amounts of antibody to be given subcutaneously. In one embodiment, the formulations described herein are administered subcutaneously.

Pharmaceutical compositions are sterile and stable under the conditions of manufacture and storage. A pharmaceutical composition can also be tested to insure it meets regulatory and industry standards for administration.

A pharmaceutical composition containing a VLA-4 binding antibody can be formulated as a solution, microemulsion, dispersion, liposome, or other ordered structure suitable to high antibody concentration. Sterile injectable solutions can be prepared by incorporating an agent described herein in the required amount in an appropriate solvent with one or a combination of ingredients enumerated above, as required, followed by filtered sterilization. Generally, dispersions are prepared by incorporating an agent described herein into a sterile vehicle that contains a basic dispersion medium and the required other ingredients from those enumerated above. The proper fluidity of a solution can be maintained, for example, by the use of a coating such as lecithin, by the maintenance of the required particle size in the case of dispersion and by the use of surfactants. Prolonged absorption of injectable compositions can be brought about by including in the composition an agent that delays absorption, for example, monostearate salts and gelatin.

Methods of Making Antibody Formulations

Formulations containing VLA-4 binding antibody formulations can be made as described in U.S. Published Application 2005/0053598, or in WO2008157356. The contents of both these applications are incorporated herein by reference.

Administration

A composition containing a VLA-4 binding antibody can be administered to a subject, e.g., a human subject, having a hematological malignancy, such as, AML, by a variety of methods. Typically, the VLA-4 binding antibody is administered parenterally, such as by subcutaneous, intravenous, intramuscular, intraarticular, intrasynovial, intrasternal, intrathecal, intrahepatic, intralesional and intracranial injection or infusion techniques. In some embodiments, a composition containing the antibody is administered intranasally.

The dosage and dose rate of a composition containing a VLA-4 binding antibody or antibody fragment effective to prevent, suppress or inhibit cell adhesion will depend on a variety of factors, such as the nature of the antibody or fragment, the size of the patient, the goal of the treatment, the nature of the pathology to be treated, the specific pharmaceutical composition used, and the judgment of the treating physician. Dosage levels of between about 0.001 and about 100 mg/kg body weight per day, e.g., between about 0.1 and about 50 mg/kg body weight per day of the active ingredient compound are useful. Typically, the VLA-4 antibody or antibody fragment, will be administered at a dose ranging between about 0.1 mg/kg body weight/day and about 20 mg/kg body weight/day, e.g., between about 0.1 mg/kg body weight/day and about 10 mg/kg body weight/day and at intervals of every 1-90 days. An antibody composition can be administered in an amount effective to provide a plasma level of antibody of at least 1 mg/ml. Optimization of dosages can be determined by administration of the binding agents, followed by assessment of the coating of VLA-4-positive cells by the agent over time after administered at a given dose in vivo.

The composition can be administered as a fixed dose, or in a mg/kg dose. Typically the administration is in a fixed dose. For example, the formulation can be administered at a fixed unit dose of between 1 mg and 500 mg (e.g., 1 mg, 50 mg, 100 mg, 150 mg, 200 mg, 250 mg, 300 mg, 350 mg, 400 mg, 450 mg) every 4 weeks (e.g., monthly), or between 50 mg and 250 mg (e.g., 75 mg, 100 mg, 150 mg, 200 mg) every two weeks, or between 25 mg and 150 mg (e.g., 50 mg, 75 mg, 100 mg, 125 mg) once a week. The formulation can also be administered in a bolus at a dose of between 1 and 8 mg/kg, e.g., about 6.0, 4.0, 3.0, 2.0, 1.0 mg/kg. Modified dose ranges include a dose that is less than 500, 400, 300, 250, 200, 150 or 100 mg/subject, typically for administration every fourth week or once a month. In one embodiment, the total dosage is 50 to 1200 mg (e.g., 100 mg, 200 mg, 300 mg, 400 mg, 500 mg, 600 mg, 700 mg, 800 mg, 900 mg, 1000 mg, of 1100 mg) every 28 days. The VLA-4 binding antibody can be administered, for example, every three to nine weeks, e.g., every fourth week, every fifth week, every sixth, every seventh week or every eighth week.

Dosage regimens can be adjusted to provide the desired response, e.g., a therapeutic response. The dose can also be chosen to reduce or avoid production of antibodies against the VLA-4 binding antibody, to achieve greater than 40, 50, 70, 75, or 80% saturation of the α4 subunit, to achieve to less than 80%, 70%, 60%, 50%, or 40% saturation of the α4 subunit, or to prevent an increase the level of circulating white blood cells.

Toxicity and therapeutic efficacy of the VLA-4 binding antibody can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Compounds that exhibit high therapeutic indices are typical.

The data obtained from cell culture assays and animal studies can be used in formulation a range of dosage for use in humans. The dosage of compositions lies generally within a range of circulating concentrations that include the ED50 with little or no toxicity. The dosage may vary within this range depending upon the dosage form employed and the route of administration utilized. For any compound used in the methods featured in the invention, the therapeutically effective dose can be estimated initially from cell culture assays. A dose may be formulated in animal models to achieve a circulating plasma concentration range of the compound or, when appropriate, of the polypeptide product of a target sequence (e.g., achieving a decreased concentration of the polypeptide) that includes the IC50 (i.e., the concentration of the test compound which achieves a half-maximal inhibition of symptoms) as determined in cell culture. Such information can be used to more accurately determine useful doses in humans. Levels in plasma may be measured, for example, by high performance liquid chromatography.

In certain embodiments, the active agent can be prepared with a carrier that will protect the antibody against rapid release, such as a controlled release formulation, including implants, and microencapsulated delivery systems. Biodegradable, biocompatible polymers can be used, such as ethylene vinyl acetate, polyanhydrides, polyglycolic acid, collagen, polyorthoesters, and polylactic acid. Many methods for the preparation of such formulations are patented or generally known. See, e.g., Sustained and Controlled Release Drug Delivery Systems, J. R. Robinson, ed., Marcel Dekker, Inc., New York, 1978.

Dosage regimens can be adjusted to provide the desired response, e.g., a therapeutic response. A “therapeutic response” is an improvement in a condition, symptom, or parameter associated with a disorder, to either a statistically significant degree or to a degree detectable to one skilled in the art.

Dosage unit form or “fixed dose” as used herein refers to physically discrete units suited as unitary dosages for the subjects to be treated; each unit contains a predetermined quantity of active antibody calculated to produce the desired therapeutic effect in association with the required pharmaceutical carrier and optionally in association with the other agent.

A pharmaceutical composition may include a “therapeutically effective amount” of a VLA-4-binding antibody, e.g., natalizumab, described herein. Such effective amounts can be determined based on the effect of the administered agent, or the combinatorial effect of an agent and secondary agent if more than one agent is used. A therapeutically effective amount of an agent may also vary according to factors such as the disease state, age, sex, and weight of the individual, and the ability of the antibody to elicit a desired response in the individual, e.g., amelioration of at least one disorder parameter, e.g., an AML parameter, or amelioration of at least one symptom of the disorder, e.g., AML. A therapeutically effective amount is also one in which any toxic or detrimental effects of the composition are outweighed by the therapeutically beneficial effects.

Devices and Kits

Compositions containing a VLA-4-binding antibody (e.g., natalizumab) for the treatment of a hematological malignancy, such as AML, can be administered with a medical device. The device can be designed with or have features such as portability, room temperature storage, and ease of use so that it can be used in emergency situations, e.g., by an untrained subject or by emergency personnel in the field, removed to medical facilities and other medical equipment. The device can include, e.g., one or more housings for storing pharmaceutical preparations that include a VLA-4-binding antibody (e.g., natalizumab), and can be configured to deliver one or more unit doses of the agent.

For example, the pharmaceutical composition can be administered with a transcutaneous delivery device, such as a syringe, including a hypodermic or multichamber syringe. In one embodiment, the device is a prefilled syringe with attached or integral needle. In other embodiments, the device is a prefilled syringe not having a needle attached. The needle can be packaged with the prefilled syringe. In one embodiment, the device is an auto-injection device, e.g., an auto-injector syringe. In another embodiment the injection device is a pen-injector. In yet another embodiment, the syringe is a staked needle syringe, luer lock syringe, or luer slip syringe. Other suitable delivery devices include stents, catheters, microneedles, and implantable controlled release devices. The composition can be administered intravenously with standard IV equipment, including, e.g., IV tubings, with or without in-line filters. In certain embodiments, the device will be a syringe for use in SC or IM administration.

Pharmaceutical compositions can be administered with medical devices. For example, pharmaceutical compositions can be administered with a needleless hypodermic injection device, such as the devices disclosed in U.S. Pat. Nos. 5,399,163, 5,383,851, 5,312,335, 5,064,413, 4,941,880, 4,790,824, or 4,596,556. Examples of well-known implants and modules include: U.S. Pat. No. 4,487,603, which discloses an implantable micro-infusion pump for dispensing medication at a controlled rate; U.S. Pat. No. 4,486,194, which discloses a therapeutic device for administering medicants through the skin; U.S. Pat. No. 4,447,233, which discloses a medication infusion pump for delivering medication at a precise infusion rate; U.S. Pat. No. 4,447,224, which discloses a variable flow implantable infusion apparatus for continuous drug delivery; U.S. Pat. No. 4,439,196, which discloses an osmotic drug delivery system having multi-chamber compartments; and U.S. Pat. No. 4,475,196, which discloses an osmotic drug delivery system. The therapeutic composition can also be in the form of a biodegradable or nonbiodegradable sustained release formulation for subcutaneous or intramuscular administration. See, e.g., U.S. Pat. Nos. 3,773,919 and 4,767,628 and PCT Application No. WO 94/15587. Continuous administration can also be achieved using an implantable or external pump. The administration can also be conducted intermittently, e.g., single daily injection, or continuously at a low dose, e.g., sustained release formulation. The delivery device can be modified to be optimally suited for administration of VLA-4 binding antibody. For example, a syringe can be siliconized to an extent that is optimal for storage and delivery of anti-VLA-4 antibody. Of course, many other such implants, delivery systems, and modules are also known.

The invention also features a device for administering a first and second agent. The device can include, e.g., one or more housings for storing pharmaceutical preparations, and can be configured to deliver unit doses of the first and second agent. The first and second agents can be stored in the same or separate compartments. For example, the device can combine the agents prior to administration. It is also possible to use different devices to administer the first and second agent.

A VLA-4-binding antibody (e.g., natalizumab) can be provided in a kit. In one embodiment, the kit includes (a) a container that contains a composition that includes a high concentration of VLA-4-binding antibody, optionally (b) a container that contains a composition that includes a second agent and optionally (c) informational material. The informational material can be descriptive, instructional, marketing or other material that relates to the methods described herein and/or the use of the agents for therapeutic benefit. In one embodiment, the kit also includes a second agent, e.g., a chemotherapeutic agent. For example, the kit includes a first container that contains a composition that includes the VLA-4-binding antibody, and a second container that includes the second agent. In one embodiment, the kit includes one or more single-use syringes pre-filled with a high concentration liquid antibody formulation described herein.

The informational material of the kits is not limited in its form. In one embodiment, the informational material can include information about production of the antibody, concentration, date of expiration, batch or production site information, and so forth. In one embodiment, the informational material relates to methods of administering the VLA-4-binding antibody (e.g., natalizumab), e.g., in a suitable dose, dosage form, or mode of administration (e.g., a dose, dosage form, or mode of administration described herein), to treat a subject who has a hematological malignancy (e.g., AML), or who is at risk for experiencing an episode associated with a hematological malignancy. The information can be provided in a variety of formats, including printed text, computer readable material, video recording, or audio recording, or information that provides a link or address to substantive material.

In addition to the agent, the composition in the kit can include other ingredients, such as a solvent or buffer, a stabilizer, or a preservative. The agent can be provided in any form, e.g., liquid, dried or lyophilized form, and in substantially pure and/or sterile form. When the agents are provided in a liquid solution, the liquid solution is, for example, an aqueous solution. When the agents are provided as a dried form, reconstitution generally is by the addition of a suitable solvent. The solvent, e.g., sterile water or buffer, can optionally be provided in the kit.

The kit can include one or more containers for the composition or compositions containing the agents. In some embodiments, the kit contains separate containers, dividers or compartments for the composition and informational material. For example, the composition can be contained in a bottle, vial, or syringe, and the informational material can be contained in a plastic sleeve or packet. In other embodiments, the separate elements of the kit are contained within a single, undivided container. For example, the composition is contained in a bottle, vial or syringe that has attached thereto the informational material in the form of a label. In some embodiments, the kit includes a plurality (e.g., a pack) of individual containers, each containing one or more unit dosage forms (e.g., a dosage form described herein) of the agents. The containers can include a combination unit dosage, e.g., a unit that includes both the VLA-4-binding antibody (e.g., natalizumab) and the second agent, e.g., a chemotherapeutic agent, in a desired ratio. For example, the kit includes a plurality of syringes, ampoules, foil packets, blister packs, or medical devices, e.g., each containing a single combination unit dose. The containers of the kits can be air tight, waterproof (e.g., impermeable to changes in moisture or evaporation), and/or light-tight.

The kit optionally includes a device suitable for administration of the composition, e.g., a syringe or other suitable delivery device. The device can be provided pre-loaded with one or both of the agents or can be empty, but suitable for loading.

Antibody Generation

Antibodies that bind to VLA-4 can be generated by immunization, e.g., using an animal, or by in vitro methods such as phage display. All or part of VLA-4 can be used as an immunogen. For example, the extracellular region of the α4 subunit can be used as an immunogen. In one embodiment, the immunized animal contains immunoglobulin producing cells with natural, human, or partially human immunoglobulin loci. In one embodiment, the non-human animal includes at least a part of a human immunoglobulin gene. For example, it is possible to engineer mouse strains deficient in mouse antibody production with large fragments of the human Ig loci. Using the hybridoma technology, antigen-specific monoclonal antibodies derived from the genes with the desired specificity may be produced and selected. See, e.g., XenoMouse™, Green et al. Nature Genetics 7:13-21 (1994), U.S. 2003-0070185, U.S. Pat. No. 5,789,650, and WO 96/34096.

Non-human antibodies to VLA-4 can also be produced, e.g., in a rodent. The non-human antibody can be humanized, e.g., as described in U.S. Pat. No. 6,602,503, EP 239 400, U.S. Pat. No. 5,693,761, and U.S. Pat. No. 6,407,213.

EP 239 400 (Winter et al.) describes altering antibodies by substitution (within a given variable region) of their complementarity determining regions (CDRs) for one species with those from another. CDR-substituted antibodies can be less likely to elicit an immune response in humans compared to true chimeric antibodies because the CDR-substituted antibodies contain considerably less non-human components. (Riechmann et al., 1988, Nature 332, 323-327; Verhoeyen et al., 1988, Science 239, 1534-1536). Typically, CDRs of a murine antibody substituted into the corresponding regions in a human antibody by using recombinant nucleic acid technology to produce sequences encoding the desired substituted antibody. Human constant region gene segments of the desired isotype (usually gamma I for CH and kappa for CL) can be added and the humanized heavy and light chain genes can be co-expressed in mammalian cells to produce soluble humanized antibody.

Queen et al., 1989 and WO 90/07861 have described a process that includes choosing human V framework regions by computer analysis for optimal protein sequence homology to the V region framework of the original murine antibody, and modeling the tertiary structure of the murine V region to visualize framework amino acid residues that are likely to interact with the murine CDRs. These murine amino acid residues are then superimposed on the homologous human framework. See also U.S. Pat. Nos. 5,693,762; 5,693,761; 5,585,089; and 5,530,101. Tempest et al., 1991, Biotechnology 9, 266-271, utilize, as standard, the V region frameworks derived from NEWM and REI heavy and light chains, respectively, for CDR-grafting without radical introduction of mouse residues. An advantage of using the Tempest et al. approach to construct NEWM and REI based humanized antibodies is that the three dimensional structures of NEWM and REI variable regions are known from X-ray crystallography and thus specific interactions between CDRs and V region framework residues can be modeled.

Non-human antibodies can be modified to include substitutions that insert human immunoglobulin sequences, e.g., consensus human amino acid residues at particular positions, e.g., at one or more (such as at least five, ten, twelve, or all) of the following positions: (in the FR of the variable domain of the light chain) 4L, 35L, 36L, 38L, 43L, 44L, 58L, 46L, 62L, 63L, 64L, 65L, 66L, 67L, 68L, 69L, 70L, 71L, 73L, 85L, 87L, 98L, and/or (in the FR of the variable domain of the heavy chain) 2H, 4H, 24H, 36H, 37H, 39H, 43H, 45H, 49H, 58H, 60H, 67H, 68H, 69H, 70H, 73H, 74H, 75H, 78H, 91H, 92H, 93H, and/or 103H (according to the Kabat numbering). See, e.g., U.S. Pat. No. 6,407,213.

Fully human monoclonal antibodies that bind to VLA-4 can be produced, e.g., using in vitro-primed human splenocytes, as described by Boerner et al., 1991, J. Immunol., 147, 86-95. They may be prepared by repertoire cloning as described by Persson et al., 1991, Proc. Nat. Acad. Sci. USA, 88: 2432-2436 or by Huang and Stollar, 1991, J. Immunol. Methods 141, 227-236; also U.S. Pat. No. 5,798,230. Large nonimmunized human phage display libraries may also be used to isolate high affinity antibodies that can be developed as human therapeutics using standard phage technology (see, e.g., Vaughan et al, 1996; Hoogenboom et al. (1998) Immunotechnology 4:1-20; and Hoogenboom et al. (2000) Immunol Today 2:371-8; U.S. 2003-0232333).

Antibody Production

Antibodies can be produced in prokaryotic and eukaryotic cells. In one embodiment, the antibodies (e.g., scFvs) are expressed in a yeast cell such as Pichia (see, e.g., Powers et al. (2001) J Immunol Methods. 251:123-35), Hanseula, or Saccharomyces.

In one embodiment, antibodies, particularly full length antibodies, e.g., IgGs, are produced in mammalian cells. Exemplary mammalian host cells for recombinant expression include Chinese Hamster Ovary (CHO cells) (including dhfr− CHO cells, described in Urlaub and Chasin (1980) Proc. Natl. Acad. Sci. USA 77:4216-4220, used with a DHFR selectable marker, e.g., as described in Kaufman and Sharp (1982) Mol. Biol. 159:601-621), lymphocytic cell lines, e.g., NS0 myeloma cells and SP2 cells, COS cells, K562, and a cell from a transgenic animal, e.g., a transgenic mammal. For example, the cell is a mammary epithelial cell.

In addition to the nucleic acid sequence encoding the immunoglobulin domain, the recombinant expression vectors may carry additional nucleic acid sequences, such as sequences that regulate replication of the vector in host cells (e.g., origins of replication) and selectable marker genes. The selectable marker gene facilitates selection of host cells into which the vector has been introduced (see e.g., U.S. Pat. Nos. 4,399,216, 4,634,665 and 5,179,017). Exemplary selectable marker genes include the dihydrofolate reductase (DHFR) gene (for use in dhfr⁻ host cells with methotrexate selection/amplification) and the neo gene (for G418 selection).

In an exemplary system for recombinant expression of an antibody (e.g., a full length antibody or an antigen-binding portion thereof), a recombinant expression vector encoding both the antibody heavy chain and the antibody light chain is introduced into dhfr− CHO cells by calcium phosphate-mediated transfection. Within the recombinant expression vector, the antibody heavy and light chain genes are each operatively linked to enhancer/promoter regulatory elements (e.g., derived from SV40, CMV, adenovirus and the like, such as a CMV enhancer/AdMLP promoter regulatory element or an SV40 enhancer/AdMLP promoter regulatory element) to drive high levels of transcription of the genes. The recombinant expression vector also carries a DHFR gene, which allows for selection of CHO cells that have been transfected with the vector using methotrexate selection/amplification. The selected transformant host cells are cultured to allow for expression of the antibody heavy and light chains and intact antibody is recovered from the culture medium. Standard molecular biology techniques are used to prepare the recombinant expression vector, to transfect the host cells, to select for transformants, to culture the host cells, and to recover the antibody from the culture medium. For example, some antibodies can be isolated by affinity chromatography with a Protein A or Protein G. For example, purified VLA-4-binding antibodies, e.g. natalizumab, can be concentrated to about 100 mg/mL to about 200 mg/mL using standard protein concentration techniques.

Antibodies may also include modifications, e.g., modifications that alter Fc function, e.g., to decrease or remove interaction with an Fc receptor or with C1q, or both. For example, the human IgG1 constant region can be mutated at one or more residues, e.g., one or more of residues 234 and 237, e.g., according to the numbering in U.S. Pat. No. 5,648,260. Other exemplary modifications include those described in U.S. Pat. No. 5,648,260.

For some antibodies that include an Fc domain, the antibody production system may be designed to synthesize antibodies in which the Fc region is glycosylated. For example, the Fc domain of IgG molecules is glycosylated at asparagine 297 in the CH2 domain. This asparagine is the site for modification with biantennary-type oligosaccharides. This glycosylation participates in effector functions mediated by Fcγ receptors and complement C1q (Burton and Woof (1992) Adv. Immunol. 51:1-84; Jefferis et al. (1998) Immunol. Rev. 163:59-76). The Fc domain can be produced in a mammalian expression system that appropriately glycosylates the residue corresponding to asparagine 297. The Fc domain can also include other eukaryotic post-translational modifications.

Antibodies can also be produced by a transgenic animal. For example, U.S. Pat. No. 5,849,992 describes a method for expressing an antibody in the mammary gland of a transgenic mammal. A transgene is constructed that includes a milk-specific promoter and nucleic acid sequences encoding the antibody of interest, e.g., an antibody described herein, and a signal sequence for secretion. The milk produced by females of such transgenic mammals includes, secreted-therein, the antibody of interest, e.g., an antibody described herein. The antibody can be purified from the milk, or for some applications, used directly.

Antibodies can be modified, e.g., with a moiety that improves its stabilization and/or retention in circulation, e.g., in blood, serum, lymph, bronchoalveolar lavage, or other tissues, e.g., by at least 1.5, 2, 5, 10, or 50 fold.

For example, a VLA-4 binding antibody can be associated with a polymer, e.g., a substantially non-antigenic polymer, such as a polyalkylene oxide or a polyethylene oxide. Suitable polymers will vary substantially by weight. Polymers having molecular number average weights ranging from about 200 to about 35,000 daltons (or about 1,000 to about 15,000, and 2,000 to about 12,500) can be used.

For example, a VLA-4 binding antibody can be conjugated to a water soluble polymer, e.g., a hydrophilic polyvinyl polymer, e.g. polyvinylalcohol or polyvinylpyrrolidone. A non-limiting list of such polymers include polyalkylene oxide homopolymers such as polyethylene glycol (PEG) or polypropylene glycols, polyoxyethylenated polyols, copolymers thereof and block copolymers thereof, provided that the water solubility of the block copolymers is maintained. Additional useful polymers include polyoxyalkylenes such as polyoxyethylene, polyoxypropylene, and block copolymers of polyoxyethylene and polyoxypropylene (Pluronics); polymethacrylates; carbomers; branched or unbranched polysaccharides that comprise the saccharide monomers D-mannose, D- and L-galactose, fucose, fructose, D-xylose, L-arabinose, D-glucuronic acid, sialic acid, D-galacturonic acid, D-mannuronic acid (e.g. polymannuronic acid, or alginic acid), D-glucosamine, D-galactosamine, D-glucose and neuraminic acid including homopolysaccharides and heteropolysaccharides such as lactose, amylopectin, starch, hydroxyethyl starch, amylose, dextrane sulfate, dextran, dextrins, glycogen, or the polysaccharide subunit of acid mucopolysaccharides, e.g. hyaluronic acid; polymers of sugar alcohols such as polysorbitol and polymannitol; heparin or heparon.

All references and publications included herein are incorporated by reference. The following examples are not intended to be limiting.

### EXAMPLES

The examples below demonstrate that the VLA-4 binding antibody natalizumab blocks VLA-4 mediated adhesion of myeloma and leukemia cell lines to ligands VCAM-1 and fibronectin, as well as to bone marrow stromal cells in co-culture experiments. Treatment of these cell lines with natalizumab is shown to disrupt survival signaling pathways and increase the sensitivity of cells to cytotoxic agents. Thus, VLA-4 adhesion is involved in the survival and chemoresistance of hematologic malignancies, and that disruption of these interactions with a VLA-4 binding antibody, such as natalizumab, is a valid therapeutic approach.

### Example 1

### VLA-4 is Expressed on Hematologic Tumor Cell Lines

The bone marrow microenvironment is involved in the development of lymphoid and myeloid progenitor cells, and also confers a protective environment to malignancies arising from these cell types. Integrin mediated adhesion interactions between bone marrow stromal cells and tumor cells confer a cytoprotective advantage in co-culture models.

As is shown below, integrin VLA-4 is widely expressed in hematologic malignancies. VLA-4 engages with fibronectin in the bone marrow matrix and vascular cell adhesion molecule-1 (VCAM-1 or CD106) on the surface of bone marrow stromal cells and activates a variety of pro-survival signaling pathways in the tumor cell.

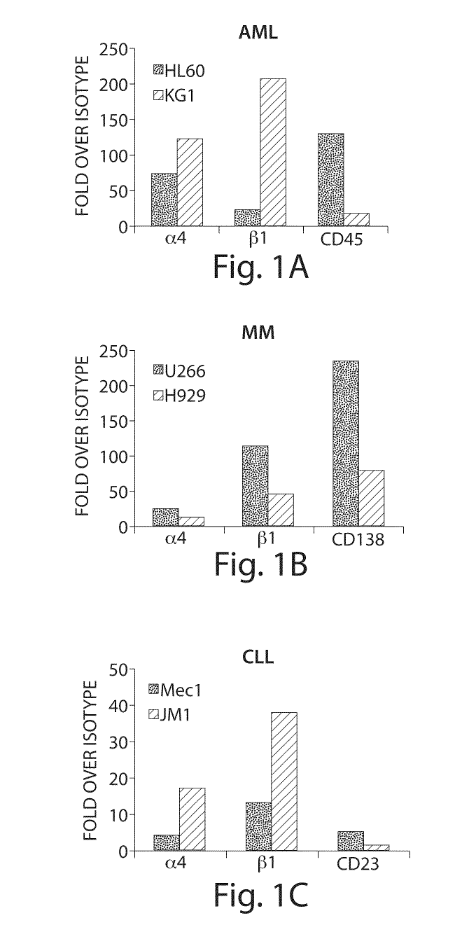

VLA-4 expression on hematologic tumor cell lines for AML, MM and CML was observed (FIGS. 1A, 1B and 1C).

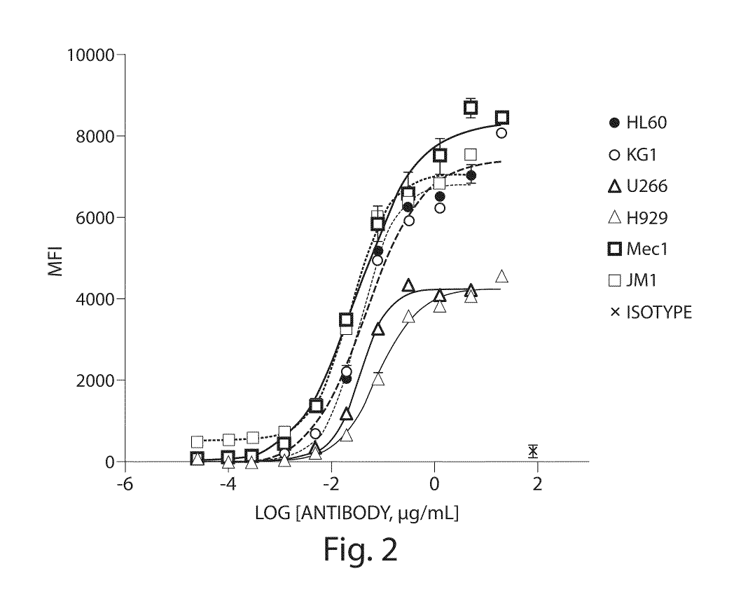

Flow cytometry experiments were performed to assess the level of VLA-4 expression on tumor cell lines, and VLA-4 expression was observed on all cell lines tested (FIG. 1).). Binding of natalizumab to AML, CLL and MM tumor cell lines was determined by flow cytometry as shown in FIG. 2. In all cases, saturable binding was observed and the calculated affinities of natalizumab (EC50 values) are shown in Table 2.

TABLE 2
Quantitation of natalizumab binding to cell lines

| | | EC₅₀ | IC₅₀ (nM) | | |
|---|---|---|---|---|---|
| | | (nM) | FN | VCAM | BMSC |
| AML | HL60 | 0.19 | 0.50 | 0.92 | 13.70 |
| | KG1 | 0.22 | 0.1 | 0.19 | nd |
| MM | U266 | 0.30 | 0.4 | 0.26 | 0.53 |
| | H929 | 0.80 | 0.4 | 0.45 | 1.74 |
| CLL | Mec1 | 0.11 | 0.39 | 0.22 | nd |
| | JM1 | 0.28 | 11.56 | 0.19 | nd |

### Example 2

### Natalizumab Inhibited Binding of Tumor Cells to VLA-4 Ligands

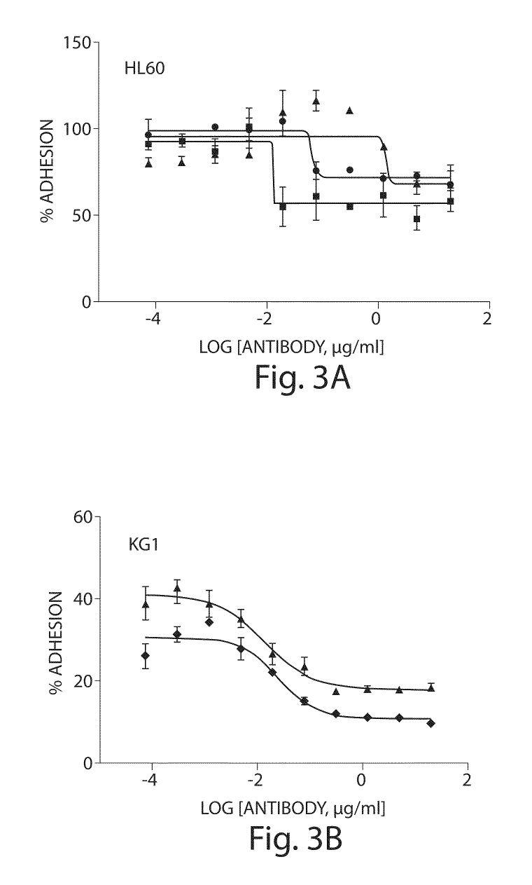

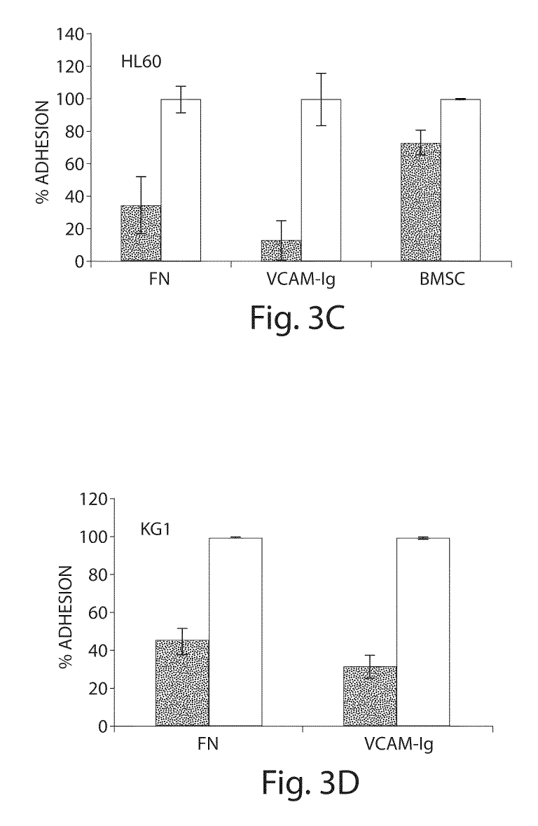

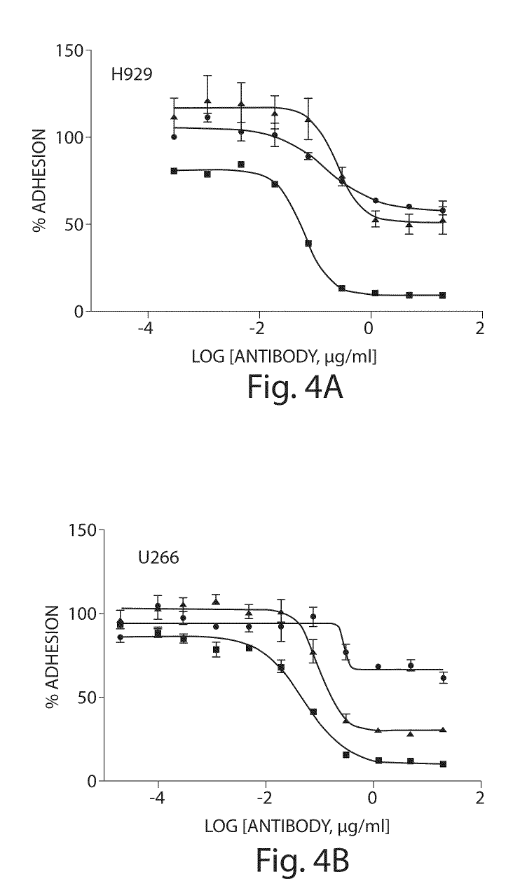

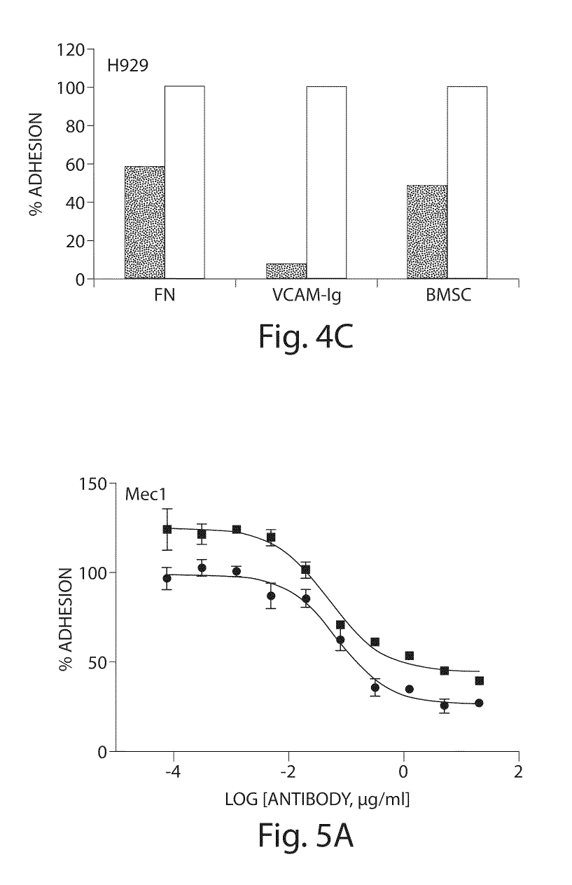

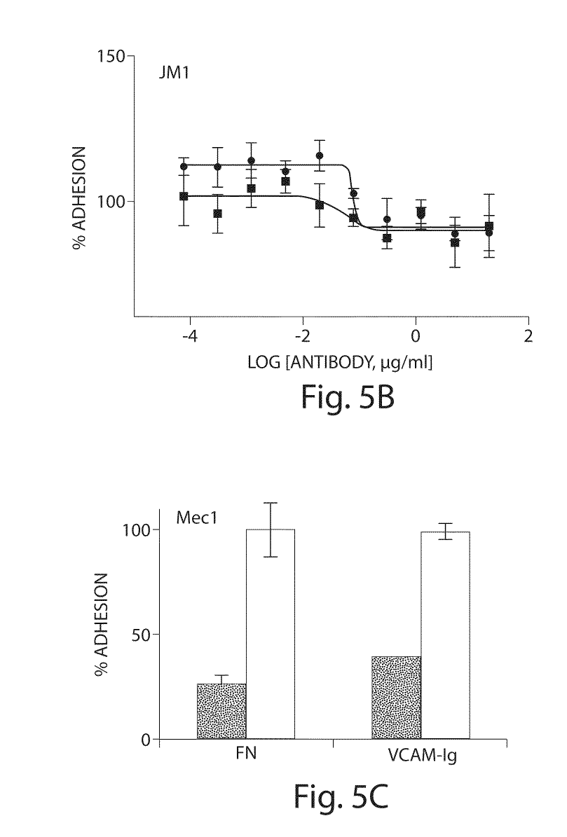

Experiments were conducted to test whether a VLA-4 antagonist can inhibit binding of tumor cells to a VLA-4 ligand. Cell lines were allowed to adhere to wells coated with fibronectin (• FN), vascular adhesion molecule-1-Ig fusion protein (▪ VCAM-Ig), or bone marrow stromal cells (▴ BMSC), in the presence of increasing concentrations of natalizumab or isotype control antibody. The results demonstrated the ability of natalizumab to inhibit adhesion of various tumor cell types to VLA-4 ligands in a dose dependent manner (FIGS. 3A, 3B, 4A, 4B, 5A and 5C). The calculated IC50 values for natalizumab inhibition of adhesion are shown in Table 2. The maximally attainable level of inhibition of tumor cell binding to VLA-4 ligands in the presence of saturating levels of natalizumab (solid bars) or isotype control (clear bars) was also assayed, and the results are shown in FIGS. 3C, 3D, 4C, and 5C. Natalizumab was shown to inhibit binding in all cell types assayed. Taken together, the above data clearly demonstrate the ability of natalizumab to bind with high affinity to VLA-4 expressing hematologic tumor cells, and to effectively inhibit VLA-4 mediated adhesion interactions in these cells.

### Example 3

### Natalizumab Abrogated Adhesion Mediated Drug Resistance of AML HL60 Cells

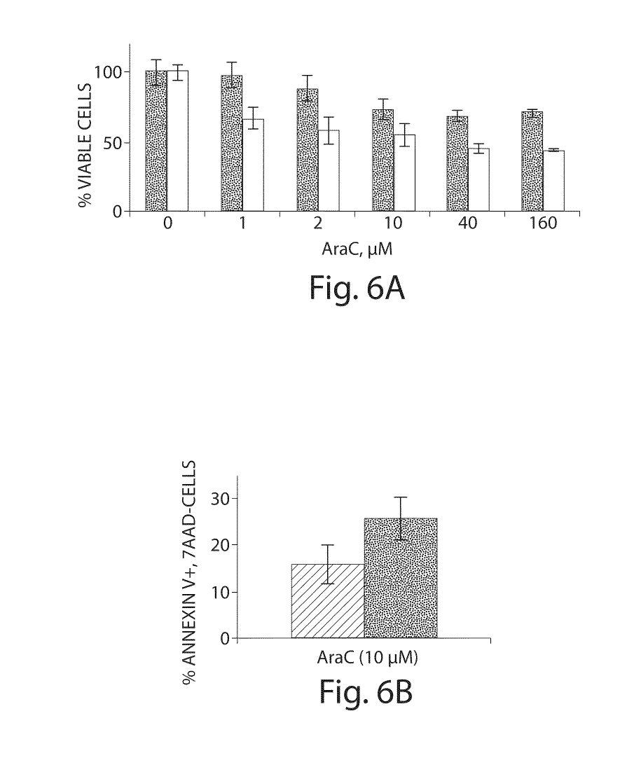

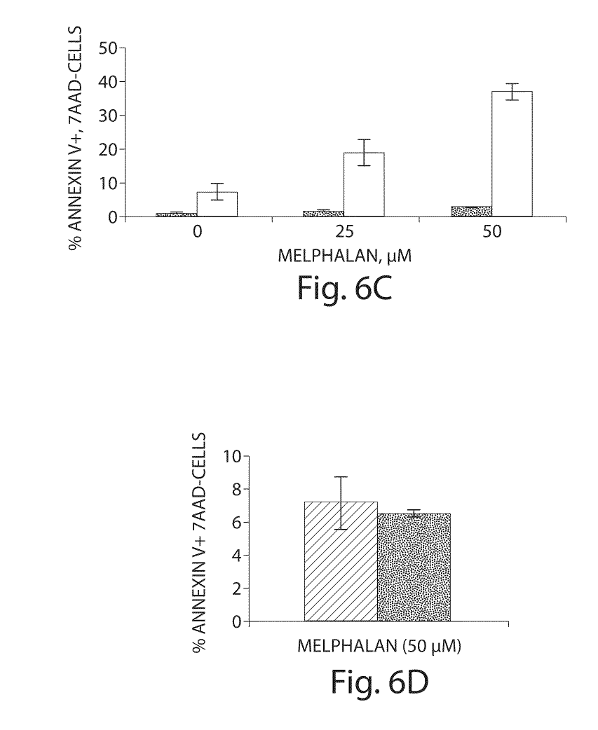

Culturing the AML cell line HL60 in the presence of BMSC conferred a protective advantage to the cells when treated with the chemotherapeutic agent ara-C, as shown in FIG. 6A. Cells were cultured for 24 hours with (▴) or without (□) BMSC, then exposed to the chemotherapy drug AraC (cytarabine) for 24 hours (FIG. 6A). Cell viability was enhanced by the presence of BMSC. When natalizumab was combined with ara-C at a concentration shown to be effective at decreasing viability in coculture in this assay (10 μM), there was an increase in the percentage of cells undergoing apoptosis, as shown in FIG. 6B. These data indicate that natalizumab can overcome the cytoprotective effect of BMSC, suggesting it could be effective in overcoming VLA-4 adhesion mediated drug resistance.

Similar experiments with the MM cell line U226 indicated that natalizumab did not have a cytoprotective effect against the drug melphalan (FIGS. 6C and 6D).

### Example 4

### Natalizumab Treatment Inhibited Co-Culture-Induced Survival Signaling Through P-STAT3

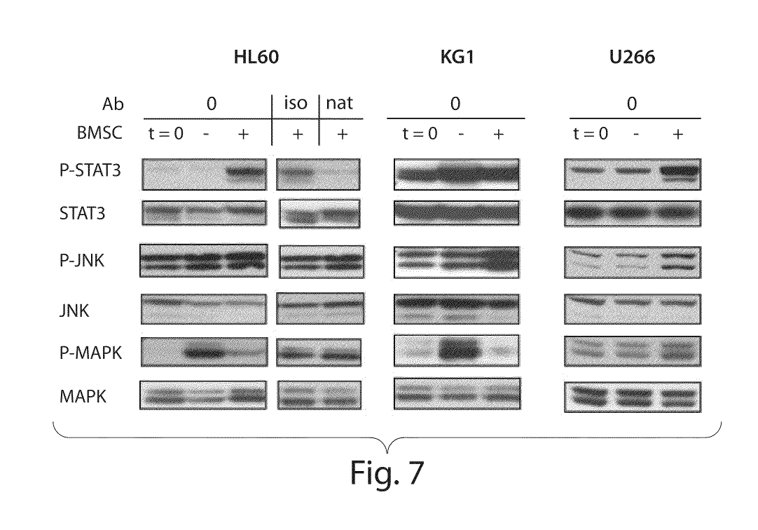

HL60, KG1 or U266 cells were grown in suspension or coculture with BMSCs and natalizumab, as indicated, for 30 minutes (HL60) or 4 hr (U266), and then the cells were separated from BMSCs, and processed for Western blot analysis to determine levels of P-STAT3, STAT3, P-JNK, JNK, P-MAPK, and MAPK (FIG. 7).

The results indicated that coculture-induced survival signaling through P-STAT3 can be inhibited by natalizumab treatment.

The results of the above experiments indicate that VLA-4 antibodies, such as natalizumab, may be effective therapeutics in hematologic malignancies.

Other embodiments are in the claims.

What is claimed is:

1. A method of treating acute myelogenous leukemia (AML) in a patient, comprising administering to the patient a therapeutically effective amount of a composition comprising an anti-alpha4 integrin antibody or antigen binding fragment thereof.

2. The method of claim 1, wherein the anti-alpha4 integrin antibody or antigen binding fragment thereof is a VLA-4 binding antibody or VLA-4 binding fragment thereof.

3. The method of claim 1, wherein the antibody or antigen binding fragment thereof is selected from the group consisting of a human antibody, a chimeric antibody, a humanized antibody and an antigen-binding Fab, Fab′, F(ab′)2 or F(v) fragment of a human, chimeric or humanized antibody.

4. The method of claim 1, wherein the composition is administered at a dosage so as to provide from about 0.1 to about 20 mg/kg body weight of the antibody or antigen binding fragment thereof.

5. The method of claim 1, wherein the antibody or antigen binding fragment thereof is a human antibody or antigen binding fragment thereof or a humanized antibody or antigen binding fragment thereof.

6. The method of claim 2, wherein the antibody or antigen binding fragment thereof is a human antibody or antigen binding fragment thereof or a humanized antibody or antigen binding fragment thereof.

7. The method of claim 3, wherein the antibody or antigen binding fragment thereof is a humanized antibody or antigen binding fragment thereof.

8. The method of claim 1, wherein the antibody or antigen-binding fragment thereof is a monoclonal antibody, or antigen-binding fragment thereof.

9. The method of claim 1, wherein the antibody or antigen-binding fragment thereof is a B epitope specific VLA-4 binding antibody or antigen-binding fragment thereof.

10. The method of claim 1, wherein the antibody is natalizumab.

11. The method of claim 1, further comprising administering a second therapeutic agent.

12. The method of claim 11, wherein the second therapeutic agent is a chemotherapeutic agent.

13. The method of claim 12, wherein the second therapeutic agent is cytarabine (Ara-C).

14. The method of claim 1, wherein the composition is administered subcutaneously or intramuscularly.


SEQUENCE LISTING (6 sequences)

SEQ ID NO: 1 - protein, 213 aa, Artificial Sequence
  Description of Artificial Sequence Synthetic polypeptide
DIQMTQSPSSLSASVGDRVTITCKTSQDINKYMAWYQQTPGKAPRLLIHYTSALQPGIPSRFSGSGSGRDYTFTISSLQPEDIATYYCLQYDNLWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 450 aa, Artificial Sequence
  Description of Artificial Sequence Synthetic polypeptide
QVQLVQSGAEVKKPGASVKVSCKASGFNIKDTYIHWVRQAPGQRLEWMGRIDPANGYTKYDPKFQGRVTITADTSASTAYMELSSLRSEDTAVYYCAREGYYGNYGVYAMDYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 3 - protein, 8 aa, Artificial Sequence
  Description of Artificial Sequence Synthetic peptide
EILDVPST

SEQ ID NO: 4 - protein, 5 aa, Artificial Sequence
  Description of Artificial Sequence Synthetic peptide
EILDV

SEQ ID NO: 5 - protein, 5 aa, Artificial Sequence
  Description of Artificial Sequence Synthetic peptide
LDVPS

SEQ ID NO: 6 - protein, 5 aa, Artificial Sequence
  Description of Artificial Sequence Synthetic Peptide
RCDPC